SEQUENCE LISTING (34 sequences)

SEQ ID NO: 1 - protein, 222 aa, Artificial Sequence
  P-LCCTL-HC Light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 2 - protein, 448 aa, Artificial Sequence
  P-LCCTL-HC Heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, Artificial Sequence
  P-LC-HCCT Light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 454 aa, Artificial Sequence
  P-LC-HCCT Heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGLPETGG

SEQ ID NO: 5 - protein, 214 aa, Artificial Sequence; identical to SEQ ID NO: 3
  P-LC-HCCTL Light chain

SEQ ID NO: 6 - protein, 456 aa, Artificial Sequence
  P-LC-HCCTL Heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGALPETGG

SEQ ID NO: 7 - protein, 220 aa, Artificial Sequence
  P-LCCT-HC Light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLPETGG

SEQ ID NO: 8 - protein, 448 aa, Artificial Sequence; identical to SEQ ID NO: 2
  P-LCCT-HC Heavy chain

SEQ ID NO: 9 - protein, 220 aa, Artificial Sequence; identical to SEQ ID NO: 7
  P-LCCT-HCCT Light chain

SEQ ID NO: 10 - protein, 454 aa, Artificial Sequence; identical to SEQ ID NO: 4
  P-LCCT-HCCT Heavy chain

SEQ ID NO: 11 - protein, 220 aa, Artificial Sequence; identical to SEQ ID NO: 7
  P-LCCT-HCCTL Light chain

SEQ ID NO: 12 - protein, 456 aa, Artificial Sequence; identical to SEQ ID NO: 6
  P-LCCT-HCCTL Heavy chain

SEQ ID NO: 13 - protein, 222 aa, Artificial Sequence; identical to SEQ ID NO: 1
  P-LCCTL-HCCT Light chain

SEQ ID NO: 14 - protein, 454 aa, Artificial Sequence; identical to SEQ ID NO: 4
  P-LCCTL-HCCT Heavy chain

SEQ ID NO: 15 - protein, 222 aa, Artificial Sequence; identical to SEQ ID NO: 1
  P-LCCTL-HCCTL Light chain

SEQ ID NO: 16 - protein, 456 aa, Artificial Sequence; identical to SEQ ID NO: 6
  P-LCCTL-HCCTL Heavy chain

SEQ ID NO: 17 - protein, 222 aa, Artificial Sequence
  hRS7 Modified Light chain
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 18 - protein, 450 aa, Artificial Sequence
  hRS7 Heavy chain
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 222 aa, Artificial Sequence
  MAAA1181a Modified Light chain
DIVMTQSPSSLSASVGDRVTITCKASQDVSTAVAWYQQKPGKQPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 20 - protein, 450 aa, Artificial Sequence
  MAAA1181a Heavy chain
QIQLVQSGAEVKKPGESVKVSCKASGYTFTTAGMQWVQQMPGKGLEWMGWINTHSGVPKYAEDFKGRVTFSLDTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 222 aa, Artificial Sequence
  Ab0052 Light chain
DIQMTQSPSSLSASVGDRVTITCKASQGINNYLSWYQQKPGKAPKSLIYRANRLVDGVPSRFSGSGSGQDYTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 22 - protein, 446 aa, Artificial Sequence
  Ab0052 Heavy chain
QVQLVQSGAEVKKPGSSVKVSCKASGYRFTDYVINWVRQAPGQGLEWMGQIYPGSDSFHYNQKFQGRATLTADKSTNTAYMELSSLRSEDTAVYYCARFFEGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 222 aa, Artificial Sequence
  Ab0053 Light chain
DIQMTQSPSSLSASVGDRVTITCKASQGINNYLSWYQQKPGKAPKSLIYRANRLVDGVPSRFSGSGSGTDYTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 24 - protein, 446 aa, Artificial Sequence; identical to SEQ ID NO: 22
  Ab0053 Heavy chain

SEQ ID NO: 25 - protein, 222 aa, Artificial Sequence
  Ab0054 Light chain
DIQMTQSPSSLSASVGDRVTITCKASQGINNYLSWYQQKPGKAPKSLIYRANRLVDGVPSRFSGSGSGTDFTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 26 - protein, 446 aa, Artificial Sequence; identical to SEQ ID NO: 22
  Ab0054 Heavy chain

SEQ ID NO: 27 - protein, 222 aa, Artificial Sequence; identical to SEQ ID NO: 25
  Ab0061 Light chain

SEQ ID NO: 28 - protein, 446 aa, Artificial Sequence
  Ab0061 Heavy chain
QVQLVQSGAEVKKPGSSVKVSCKASGYRFTDYVINWVRQAPGQGLEWMGQIYPGSDTFHYNQKFQGRATLTADKSTNTAYMELSSLRSEDTAVYYCARFFEGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 222 aa, Artificial Sequence; identical to SEQ ID NO: 25
  Ab0062 Light chain

SEQ ID NO: 30 - protein, 446 aa, Artificial Sequence
  Ab0062 Heavy chain
QVQLVQSGAEVKKPGSSVKVSCKASGYRFTDYVINWVRQAPGQGLEWMGQIYPGSDAFHYNQKFQGRATLTADKSTNTAYMELSSLRSEDTAVYYCARFFEGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 222 aa, Artificial Sequence
  Ab0063 Light chain
DIQMTQSPSSLSASVGDRVTITCKASQGINNYLSWYQQKPGKAPKSLIYRANRLVSGVPSRFSGSGSGTDFTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 32 - protein, 446 aa, Artificial Sequence; identical to SEQ ID NO: 22
  Ab0063 Heavy chain

SEQ ID NO: 33 - protein, 222 aa, Artificial Sequence; identical to SEQ ID NO: 31
  Ab0064 Light chain

SEQ ID NO: 34 - protein, 446 aa, Artificial Sequence; identical to SEQ ID NO: 28
  Ab0064 Heavy chain

(19) United States
(12) Patent Application Publication
(10) Pub. No.: US 2023/0256108 A1
(43) Pub. Date: Aug. 17, 2023

(54) A DRUG CONJUGATE AND APPLICATIONS THEREOF

(71) Applicant: GENEQUANTUM HEALTHCARE (SUZHOU) CO., LTD., Suzhou, Jingsu (CN)

(72) Inventors: Gang QIN, Suzhou (CN); Lu JIANG, Suzhou (CN); Lili SHI, Suzhou (CN); Jinduo YUAN, Suzhou (CN); Chong LIU, Suzhou (CN); Lehua HU, Suzhou (CN)

(21) Appl. No.: 17/758,268

(22) PCT Filed: Dec. 31, 2020
(86) PCT No.: PCT/CN2020/141928

§ 371 Date: Jun. 30, 2022

(30) Foreign Application Priority Data
Dec. 31, 2019 (WO) PCT/CN2019/130859

(51) Int. Cl. A61K 47/65; A61K 47/68; A61P 35/00

(52) U.S. Cl. CPC A61K 47/65; A61K 47/6803; A61K 47/6851; A61K 47/6889; A61P 35/00

(57) ABSTRACT

A linker molecule for targeting molecule-drug conjugate, has the structure of formula (I): A1p-D1q-Y-Lk-W-A2q-D2p (I). A conjugate has the structure of formula (III): A-((compound of formula (1))-PL ₜ) _{z} (III). The conjugate can be used in the manufacture of a medicament for treating a disease.

### TECHNICAL FIELD

The present disclosure relates to the biopharmaceutical field, particularly to a linker for targeting molecule-drug conjugate, and the corresponding conjugate, the preparing process and use thereof.

### BACKGROUND

Targeting molecule-drug conjugates are a class of targeting drugs developed in recent years, which are formed by coupling a payload with a targeting molecule through a linking unit/linker. The present targeting molecule-drug conjugates that are approved by FDA are mainly antibody-drug conjugates (ADCs), including Gemtuzumab ozogamicin (Mylotarg®, Pfizer/Wyeth), Inotuzumab ozogamicin (Besponsa®, Pfizer), Brentuximab vedotin (Adcetris®, Seattle Genetics), Ado-trastuzumab emtansine (Kadcyla®, Genentech/Roche) and Polatuzumab vedotin-piiq (Polivy®, Roche). The commercially available ADCs and most of the ADCs in clinical trials are prepared by chemical coupling.

Generally, chemical coupling is not site specific. The coupling reaction between the linking unit and the antibody occurs very randomly, resulting in great difference of the number and location of the conjugated payloads among the individual antibody molecules and the resulting ADCs show high heterogeneity. Although the drug antibody ratio (DAR) can be controlled within a certain range by processing control, preparation of an ADC molecule actually results in a mixture with different structures/components. This heterogeneity not only poses great challenges to drug production and quality control, but also has a great impact on the activity, biodistribution, metabolism, and safety.

Another defect is off-target release which causes toxicity to normal tissues, and reduces the number of ADCs at the target, resulting in reduced efficacy. More than half of the ADCs which are commercially available or in clinical trials use a thiosuccinimide structure (thiosuccinimide linkage) to couple the small molecule drug with the targeting antibody or protein. The thiosuccinimide structure is formed by the reaction of a thiol group and a maleimide. However, the thiosuccinimide linkage is not stable. In organisms, reverse Michael addition or exchange with other thiol groups may occur, directly leading to the fall-off of the cytotoxin from the ADC and resulting in off-target toxicity. This defect affects the safety and limits the clinical application.

### SUMMARY

In one aspect, provide is a compound of formula (I):

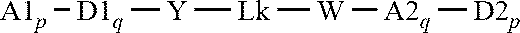

wherein,
D1 and D2 are independently a moiety comprising a recognition sequence of the ligase acceptor or donor substrate;
A1 and A2 are independently a moiety comprising a reactive group which can be coupled with a payload;
Lk is L₁—L₂—L₃;
L₁ and L₃ are each independently selected from the group consisting of:
—CH₂—, —NH—, —(CO)—, —NH(CO)—, —(CO)NH—; and combination of a C₁₋₄ alkylene with one of the following groups: —CH₂—, —NH—, —(CO)—, —NH(CO)—, —(CO)NH—;
L₂ is absent or is a C₇₋₃₄ alkylene, and wherein one or more (—CH₂—) structures in the alkylene is optionally replaced by —O—;
L₁, L₂ and L₃ are each optionally and independently substituted with 1, 2 or 3 substituents selected from —OR₁ and —NR₁R₂;
R₁ and R₂ are each independently selected from the group consisting of hydrogen, -C₁₋₆ alkyl, -(CO)-C₁₋₆ alkyl and -S(=O)₂-C₁₋₆ alkyl;
Y and W are each independently absent or selected from the group consisting of a cleavable sequence, spacer Sp1, and the combination thereof;
the cleavable sequence comprises an amino acid sequence which can be cleaved by enzyme, and the cleavable sequence comprises 1-10 amino acids;
Sp1 is selected from the group consisting of a spacer sequence containing 1-20 amino acids, PAB, and the combination thereof;
p is 0 or 1, q is 0 or 1, provided that p and q are different.

In another aspect, provided is a compound having the structure of formula (II)

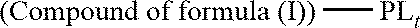

wherein
PL is a Payload which is linked to the A1 or A2 moiety of the compound of formula (I);
t is an integer of 1 to 20.

In yet another aspect, provided is a conjugate having the structure of formula (III)

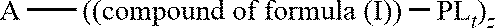

wherein
PL is a charge (Payload) which is linked to the A1 or A2 moiety of the compound of formula (I);
A is a targeting molecule which is linked to the D1 or D2 moiety of the compound of formula (I);
z is an integer of 1 to 20;
t is an integer of 1 to 20.

In another aspect, provided is a pharmaceutical composition comprising a conjugate of the present disclosure, and use of a conjugate of the present disclosure or a pharmaceutical composition of the present disclosure in the manufacture of a medicament for treating a disease, disorder or condition.

### BRIEF DESCRIPTION OF THE DRAWINGS

FIG. 1: SDS-PAGE results of the modified antibodies P-LCCTL-HC and DG102.

FIG. 2: Affinities of antibody Pertuzumab, P-LCCT_{L}-HC and DG102 to HCC1954 cells (FACS assay).

FIG. 3: Affinities of antibody Pertuzumab, P-LCCT_{L}-HC and DG102 to SK-BR-3 cells (FACS assay).

FIG. 4: Affinities of antibody Pertuzumab, P-LCCT_{L}-HC and DG102 to SK-BR-3 cells (FACS assay).

FIG. 5: The effect of ADC on MDA-MB-231 cell proliferation.

FIG. 6: The effect of ADC on MDA-MB-468 cell proliferation.

FIG. 7: DG102 effect on tumor volume (n = 6, Mean ± SEM.).

FIG. 8: DG202 effect on tumor volume (n = 6, Mean ± SEM.).

FIG. 9: DG1002 effect on tumor volume (n = 6, Mean ± SEM.).

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patents applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “i is an integer of 2 to 20” means that i is any integer of 2 to 20, for example, i can be 2, 3, 4, 5, 6, 7, 8, 9, 10, 11, 12, 13, 14, 15, 16, 17, 18, 19 or 20. Other similar expressions should also be understood in a similar manner.

Unless otherwise stated herein, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within ± 10% of a specified value, or a wider range.

The term “stoichiometric ratio” means matching various substances according to a certain amount by weight. For example, in the present disclosure, the active ingredient is mixed with a filler, a binder, and a lubricant in a designated weight ratio.

The term “optional” or “optionally” means the event described subsequent thereto may, but not necessarily happen, and the description includes the cases wherein the said event or circumstance happens or does not happen.

The expression “comprising” or similar expressions “including,” “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

The term “targeting molecule” refers to a molecule that has an affinity for a particular target (e.g., receptor, cell surface protein, cytokine, etc.). A targeting molecule can deliver the payload to a specific site in vivo through targeted delivery. A targeting molecule can recognize one or more targets. The specific target sites are defined by the targets it recognizes. For example, a targeting molecule that targets a receptor can deliver a cytotoxin to a site containing a large number of the receptor. Examples of targeting molecules include, but are not limited to antibodies, antibody fragments, binding proteins for a given antigen, antibody mimics, scaffold proteins having affinity for a given target, ligands, and the like.

As used herein, the term “antibody” is used in a broad way and particularly includes intact monoclonal antibodies, polyclonal antibodies, monospecific antibodies, multispecific antibodies (e.g., bispecific antibodies), and antibody fragments, as long as they have the desired biological activity. The antibody may be of any subtype (such as IgG, IgE, IgM, IgD, and IgA) or subclass, and may be derived from any suitable species. In some embodiments, the antibody is of human or murine origin. The antibody may also be a fully human antibody, humanized antibody or chimeric antibody prepared by recombinant methods.

Monoclonal antibodies are used herein to refer to antibodies obtained from a substantially homogeneous population of antibodies, i.e., the individual antibodies constituting the population are identical except for a small number of possible natural mutations. Monoclonal antibodies are highly specific for a single antigenic site. The word “monoclonal” refers to that the characteristics of the antibody are derived from a substantially homogeneous population of antibodies and are not to be construed as requiring some particular methods to produce the antibody.

An intact antibody or full-length antibody essentially comprises the antigen-binding variable region(s) as well as the light chain constant region(s) (C_{L}) and heavy chain constant region(s) (C_{H}), which could include C_{H}1, C_{H}2, C_{H}3 and C_{H}4, depending on the subtype of the antibody. An antigen-biding variable region (also known as a fragment variable region, Fv fragment) typically comprises a light chain variable region (V_{L}) and a heavy chain variable region (V_{H}). A constant region can be a constant region with a native sequence (such as a constant region with a human native sequence) or an amino acid sequence variant thereof. The variable region recognizes and interacts with the target antigen. The constant region can be recognized by and interacts with the immune system.

An antibody fragment may comprise a portion of an intact antibody, preferably its antigen binding region or variable region. Examples of antibody fragments include Fab, Fab′, F(ab′)₂, Fd fragment consisting of V_{H} and C_{H}1 domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a V_{L} and a C_{L}) and another chain, wherein the said other chain comprises a variable domain of the heavy chain (V_{H}) and a constant region domain of the heavy chain (C_{H}1). The F(ab′)₂ fragment is an antibody fragment obtained by pepsin digestion of an immunoglobulin at pH 4.0-4.5, or a fragment having the same structure produced by, for example, recombinant expression. The F(ab′)₂ fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)₂ fragment (one heavy chain and one light chain). The antibody fragment may comprise a plurality of chains joined together, for example, via a disulfide bond and/or via a peptide linker. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at least or about 200 amino acids. An antigen-binding fragment can include any antibody fragment that, when inserted into an antibody framework (e.g., by substitution of the corresponding region), can result in an antibody that immunospecifically binds to the antigen.

Antibodies according to the present disclosure can be preprared using techniques well known in the art, such as the following techniques or a combination thereof: recombinant techniques, phage display techniques, synthetic techniques, or other techniques known in the art. For example, a genetically engineered recombinant antibody (or antibody mimic) can be expressed by a suitable culture system (e.g., E. coli or mammalian cells). The engineering can refer to, for example, the introduction of a ligase-specific recognition sequence at its terminals.

HER2 refers to human epidermal growth factor receptor-2, which belongs to the epidermal growth factor (EGFR) receptor tyrosine kinase family. In the present application, the terms ErbB2 and HER2 have the same meaning and can be used interchangeably.

TROP2 is a transmembrane glycoprotein encoded by the Tacstd2 gene. TROP2 is an intracellular calcium signal transducer and is overexpressed in a variety of tumors.

As used herein, the term “targeting molecule-drug conjugate” is referred to as “conjugate”. Examples of conjugates include, but are not limited to, antibody-drug conjugates.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

Cytotoxin refers to a substance that inhibits or prevents the expression activity of a cell, cellular function, and/or causes destruction of cells. The cytotoxins currently used in ADCs are more toxic than chemotherapeutic drugs. Examples of cytotoxins include, but are not limited to, drugs that target the following targets: microtubule cytoskeleton, DNA, RNA, kinesin-mediated protein transport, regulation of apoptosis. The drug that targets microtubule cytoskeleton may be, for example, a microtubule-stabilizing agent or a tubulin polymerization inhibitor. Examples of microtubule-stabilizing agents include but are not limited to taxanes. Examples of tubulin polymerization inhibitors include but are not limited to maytansinoids, auristatins, vinblastines, colchicines, and dolastatins. The DNA-targeting drug can be, for example, a drug that directly disrupts the DNA structure or a topoisomerase inhibitor. Examples of drugs that directly disrupt DNA structure include but are not limited to DNA double strand breakers, DNA alkylating agents, DNA intercalators. The DNA double strand breakers can be, for example, an enediyne antibiotic, including but not limited to dynemicin, esperamicin, neocarzinostatin, uncialamycin, and the like. The DNA alkylating agent may be, for example, a DNA bis-alkylator (i.e. DNA-cross linker) or a DNA mono-alkylator. Examples of DNA alkylating agents include but are not limited to pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound, and the like. Examples of topoisomerase inhibitors include but are not limited to camptothecins and anthracyclines. The RNA-targeting drug may be, for example, a drug that inhibits splicing, and examples thereof include but are not limited to pladienolide. Drugs that target kinesin-mediated protein transport can be, for example, mitotic kinesin inhibitors including, but not limited to, kinesin spindle protein (KSP) inhibitors.

A spacer is a structure that is located between different structural modules and can spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid such as Leu, Gln, etc., a sequence containing a plurality of amino acids, for example, a sequence containing two amino acids such as GA, etc., or, for example, GGGS, GGGGSGGGGS, etc.. Other examples of spacers include, for example, self-immolative spacers such as PAB (p-aminobenzyl), and the like.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers. A bivalent radical refers to a group obtained from the corresponding monovalent radical by removing one hydrogen atom from a carbon atom with free valence electron(s). A bivalent radical have two connecting sites which are connected to the rest of the molecule. For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃H₆ —), butylene (—C₄H₈—), pentylene (—C₅H₁₀—), hexylene (—C₆H₁₂—), 1-methylethylene (—CH(CH₃)CH₂—), 2-methylethylene (—CH2CH(CH3)—), methylpropylene, ethylpropylene, and the like.

As used herein, when a group is combined with another group, the connection of the groups may be linear or branched, provided that a chemically stable structure is formed. The structure formed by such a combination can be connected to other moieties of the molecule via any suitable atom in the structure, preferably via a designated chemical bond. For example, when describing a combination of a C₁₋₄ alkylene with one of the groups including —CH₂—, —NH—, —(CO)—, —NH(CO)—, —(CO)NH—, the C₁₋₄ alkylene may form a linear connection with the above groups, such as C₁₋₄ alkylene-CH2-, C₁₋₄ alkylene-NH-, C₁₋₄ alkylene-(CO)-, C₁₋₄ alkylene-NH(CO)-, C₁₋₄ alkylene-(CO)NH-, -CH₂-C₁₋₄ alkylene, -NH-C₁₋₄ alkylene, -(CO)-C₁₋₄ alkylene, -NH(CO)-C₁₋₄ alkylene, -(CO)NH-C₁₋₄ alkylene. The resulting bivalent structure can be further connected to other moieties of the molecule.

As used herein, the expressions “antibody-conjugated drug” and “antibody-drug conjugate” has the same meaning.

### Compound of Formula (I)

In one aspect, provide is a compound of formula (I):

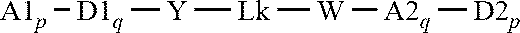

wherein,
D1 and D2 are independently a moiety comprising a recognition sequence of the ligase acceptor or donor substrate;
A1 and A2 are independently a moiety comprising a reactive group which can be coupled with a payload;
Lk is L₁—L₂—L₃;
L₁ and L₃ are each independently selected from the group consisting of:
—CH₂—, —NH—, —(CO)—, —NH(CO)—, —(CO)NH—; and combination of a C₁₋₄ alkylene with one of the following groups: —CH₂—, —NH—, —(CO)—, —NH(CO)—, —(CO)NH—;
L₂ is absent or is a C₇₋₃₄ alkylene, and wherein one or more (—CH₂—) structures in the alkylene is optionally replaced by —O—;
L₁, L₂ and L₃ are each optionally and independently substituted with 1, 2 or 3 substituents selected from —OR₁ and —NR₁R₂;
R₁ and R₂ are each independently selected from the group consisting of hydrogen, -C₁₋₆ alkyl, -(CO)-C₁₋₆ alkyl and -S(=O)₂-C₁₋₆ alkyl;
Y and W are each independently absent or selected from the group consisting of a cleavable sequence, spacer Sp1, and the combination thereof;
the cleavable sequence comprises an amino acid sequence which can be cleaved by enzyme, and the cleavable sequence comprises 1-10 amino acids;
Sp1 is selected from the group consisting of a spacer sequence containing 1-20 amino acids, PAB, and the combination thereof;
p is 0 or 1, q is 0 or 1, provided that p and q are different.

In one embodiment, L₁, L₂ and L₃ are independently substituted with 1, 2, or 3 substituents selected from -OR₁ and -NR₁R₂. Substitutions occur, for example, on (-CH₃),

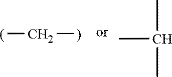

structure, especially on (—CH₂—).

In one embodiment, L¹ is —NH—, or is a combination of a C₁₋₄ alkylene with —NH—. In another embodiment, L¹ is —(CO)—, or is a combination of a C₁₋₄ alkylene with —(CO)—.

In one embodiment, L³ is —NH—, or is a combination of a C₁₋₄ alkylene with —NH—. In another embodiment, L³ is —(CO)—, or is a combination of a C₁₋₄ alkylene with —(CO)—.

In one embodiment, L₂ is a C₇₋₃₄ alkylene, wherein the alkylene is a straight or a branched alkylene group, and optionally one or more of the (—CH₂—) structures in the alkylene can be replaced by —O—, and the alkylene is optionally substituted with 1, 2 or 3 substituents selected from —OR₁ and —NR₁R₂. In yet another embodiment, L₂ is selected from groups optionally substituted with 1, 2 or 3 substituents selected from —OR₁ and —NR₁R₂, wherein the said groups are as follows: methylene, ethylene, propylene, butylene, pentylene, hexylene, 1-methylethylene, 2-methylethylene, 2-methylpropylene and 2-ethylpropylene.

In another embodiment, L₂ is -(C₂H₄-O)i-C₁₋₄ alkylene; i is an integer of 2 to 10. “—(C₂H₄—O)ᵢ—” represents a structure formed by polymerization of PEG units, wherein i indicates the number of PEG units. In another embodiment, L₂ is -(C₂H₄-O)ᵢ-C₁₋₂ alkylene. In a particular embodiment, L₂ is —(C₂H₄—O)i—C₂H₄—. In another embodiment, L₂ is C₁₋₄ alkylene—(O—C₂H₄)ᵢ. In another embodiment, L₂ is C₁-₂ alkylene—(O—C₂H₄)ᵢ. In a particular embodiment, L₂ is —C₂H₄—(O—C₂H₄)ᵢ—. In one embodiment, i is selected from the following values: 2-10, 2-8, 2-6, 2-4 or 4-6. In a particular embodiment, i is 4.

In one embodiment, Y and W are each independently absent or selected from the group consisting of a cleavable sequence, spacer Sp1, and the combination thereof. In a particular embodiment, Y is absent. In another particular embodiment, W is absent. In yet another particular embodiment, Y and W are both absent. In one embodiment, the cleavable sequence comprises an amino acid sequence that can be recognized as enzyme substrate and can be cleaved by the enzyme. In a particular embodiment, the cleavable sequence can be enzymatically cleaved in the cell, especially in the lysosome of the cell. In another particular embodiment, the cleavable sequence can be cleaved by protease, in particular by cathepsins. In yet another particular embodiment, the cleavable sequence can be cleaved by glutaminase. In one embodiment, the cleavable sequence is selected from the group consisting of a cathepsin restriction site, a glutaminase restriction site, and combinations thereof. In one embodiment, the cleavable sequence is selected from Phe-Lys, Val-Cit, Val-Lys, GLy-Phe-Leu-Gly, Ala-Leu-Ala-Leu and the combination thereof.

In one embodiment, Y and W are each independently absent or selected from spacer Sp1. In another embodiment, Sp1 is a spacer sequence comprising 1-10, preferably 1-6, more preferably 1-4 amino acids. In a particular embodiment, Sp1 is Leu. In another particular embodiment, Sp1 is Gln. In one embodiment, Sp1 is PAB. In yet another embodiment, Y and W are each independently selected from the group consisting of Phe-Lys-PAB, Val-Cit-PAB, and Val-Lys-PAB.

In one embodiment, the amino acids comprised by Y and/or W may be natural or unnatural. In a particular embodiment, Y is absent, or is amino acid fragment 1. Amino acid fragment 1 comprises 1-30 natural or unnatural amino acids, which are each independently the same or different. And amino acid fragment 1 is selected from the group consisting of: a cleavable sequence comprising 1-10 amino acids, a spacer sequence comprising 1-20 amino acids, and the combination thereof. In another particular embodiment, W is absent, or is amino acid fragment 2. Amino acid fragment 2 comprises 1-30 natural or unnatural amino acids, which are each independently the same or different. And amino acid fragment 2 is selected from the group consisting of: a cleavable sequence comprising 1-10 amino acids, a spacer sequence comprising 1-20 amino acids, and the combination thereof.

In one embodiment, p=0, q=1, the structure of the compound of formula (I) is as shown in the following formula (1-1):

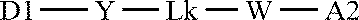

wherein, A2, D1, Y, Lk, and W are as defined in formula (I), respectively.

In another embodiment, p = 1, q = 0, the structure of the compound of formula (I) is as shown in the following formula (I-2):

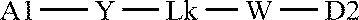

wherein, A1, D2, Y, Lk and W are as defined in formula (I), respectively.

### Moiety Comprising Recognition Sequence of the Ligase Acceptor or Donor Substrate

In one embodiment, the ligase is a transpeptidase. In one embodiment, the ligase is selected from the group consisting of a natural transpeptidase, an unnatural transpeptidase, variants thereof, and the combination thereof. Unnatural transpeptidase enzymes can be, but are not limited to, those obtained by engineering of natural transpeptidase. In a preferred embodiment, the ligase is selected from the group consisting of a natural Sortase, an unnatural Sortase, and the combination thereof. The species of natural Sortase include Sortase A, Sortase B, Sortase C, Sortase D, Sortase L. plantarum, etc. (US20110321183A1). The type of ligase corresponds to the ligase recognition sequence and is thereby used to achieve specific coupling between different molecules or structural fragments. In one embodiment, the recognition sequence of the ligase acceptor substrate is selected from the group consisting of oligomeric glycine, oligomeric alanine, and a mixture of oligomeric glycine/alanine having a degree of polymerization of 3-10. In a particular embodiment, the recognition sequence of the ligase acceptor substrate is Gₙ, wherein G is glycine (Gly), and n is an integer of 3 to 10. In another particular embodiment, the ligase is Sortase A from Staphylococcus aureus. Accordingly, the ligase recognition sequence may be the typical recognition sequence LPXTG of the enzyme. In yet another particular embodiment, the recognition sequence of the ligase donor substrate is LPXTGJ, and the recognition sequence of the ligase acceptor substrate is Gₙ, wherein X can be any single amino acid that is natural or unnatural; J is absent, or is an amino acid fragment comprising 1-10 amino acids, optionally labeled. In one embodiment, J is absent. In yet another embodiment, J is an amino acid fragment comprising 1-10 amino acids, wherein each amino acid is independently any natural or unnatural amino acid. In another embodiment, J is Gₘ, wherein m is an integer of 1 to 10. In yet another particular embodiment, the recognition sequence of the ligase donor substrate is LPETG. In another particular embodiment, the recognition sequence of the ligase donor substrate is LPETGG. In one embodiment, the ligase is Sortase B from Staphylococcus aureus and the corresponding donor substrate recognition sequence can be NPQTN. In another embodiment, the ligase is Sortase B from Bacillus anthracis and the corresponding donor substrate recognition sequence can be NPKTG. In yet another embodiment, the ligase is Sortase A from Streptococcus pyogenes and the corresponding donor substrate recognition sequence can be LPXTGJ, wherein J is as defined above. In another embodiment, the ligase is Sortase subfamily 5 from Streptomyces coelicolor, and the corresponding donor substrate recognition sequence can be LAXTG. In yet another embodiment, the ligase is Sortase A from Lactobacillus plantarum and the corresponding donor substrate recognition sequence can be LPQTSEQ. The ligase recognition sequence can also be other totally new recognition sequence for transpeptidase optimized by manual screening.

### Moiety Comprising Reactive Group

In one embodiment, A1 and A2 in formula (I) are each independently selected from the group consisting of amino compound, maleimide and derivative thereof, thiol compound, pyridyldithiol compound, haloacetic acid (haloacetylic acid), isocyanate. In another embodiment, the reactive groups in A1 and A2 are each independently selected from the group consisting of: amino group, maleimide group, thiol group, pyridyldithio group, haloacetyl group, and isocyanate group. In yet another embodiment, according to the structure of the reactive group therein, A1 and A2 can each independently covalently couple with a Michael acceptor (the acceptor molecule of Michael addition) through a disulfide bond, a thioether bond, a thioester bond, or a urethane bond. In a particular embodiment, A1 and A2 are each independently selected from optionally derivatized cysteines.

In another particular embodiment, A1 and A2 are each independently selected from optionally derivatized cysteines. In a preferred embodiment, the derivatization of cysteine is selected from the group consisting of: 1) amidation of the carboxyl group, the resulting amide NH₂ being optionally substituted with a C₁₋₆ alkyl group; 2) acylation of the amino group; and 3) linkage of the carboxyl group and/or the amino group to an amino acid fragment comprising 1-10 amino acids or a nucleotide fragment comprising 1-10 nucleotides, wherein the amino acid fragment is preferably Gly. In a particular embodiment, the derivatization of cysteine refers to amidation or linkage to glycine for the carboxyl group of cysteine.

In one embodiment, A2 is

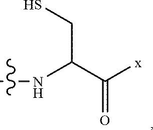

wherein x is selected from the group consisting of hydrogen, OH, NH₂, an amino acid fragment comprising 1-10 amino acids, and a nucleotide fragment comprising 1-10 nucleotides. In one embodiment, A1 is

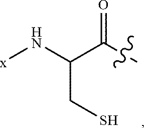

wherein x is selected from the group consisting of hydrogen, an amino acid fragment comprising 1-10 amino acids, and a nucleotide fragment comprising 1-10 nucleotides. In one embodiment, acylation of the amino group refers to the substitution with a C₁₋₆ alkylcarbonyl group for the amino group of cysteine.

The linking unit of formula (I-1), wherein D1 is Gₙ, G is glycine, A2 is

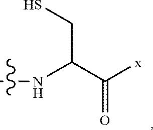

and the structure of the compound of formula (I-1) is as shown in the following formula (I-1-1):

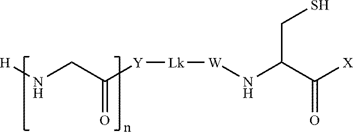

wherein n is an integer of 3 to 10;
x is selected from the group consisting of hydrogen, OH, NH₂, an amino acid fragment comprising 1-10 amino acids, a nucleotide fragment comprising 1-10 nucleotides;
Y, Lk and W are as defined in formula (I), respectively.

In a preferred embodiment, in formula (I-1-1), x is selected from OH, NH₂ and Gly.

In a particular embodiment, in formula (I-1-1), both Y and W are absent, n=3, Lk is L₁-L₂-L₃, L₁ is —NH—, L₃ is —(CO)—, L₂ is —(C₂H₄—O)ᵢ—C₂H₄—, i=4, and x is NH₂, and the structure of the linking unit is as follows (linking unit LU102):

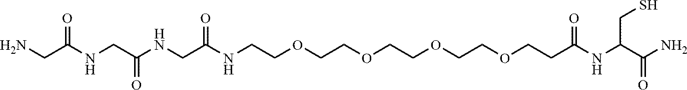 [SMILES: NCC(=O)NCC(=O)NCC(=O)NCCOCCOCCOCCOCCC(=O)NC(CS)C(N)=O]

In a particular embodiment, in formula (I-1-1), both Y and W are absent, n=3, Lk is L₁-L₂-L₃, L₁ is —NH—, L₃ is —(CO)—, L₂ is —(C₂H₄—O)ᵢ—C₂H₄—, i=4, x is OH, and the structure of the linking unit is as follows (linking unit LU106):

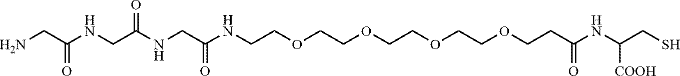 [SMILES: NCC(=O)NCC(=O)NCC(=O)NCCOCCOCCOCCOCCC(=O)NC(CS)C(=O)O]

In a particular embodiment, in formula (I-1-1), W is absent, Y is L, L is leucine (Leu), n=3, Lk is L₁-L₂-L₃, L₁ is —NH—, L₃ is —(CO)—, L₂ is —(C₂H₄—O)ᵢ—C₂H₄—, i=4, x is NH₂, and the structure of the linking unit is as follows (linking unit LU107):

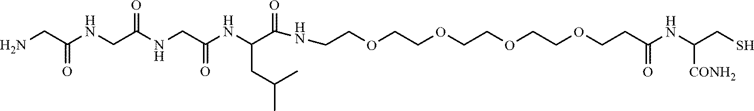 [SMILES: CC(C)CC(NC(=O)CNC(=O)CNC(=O)CN)C(=O)NCCOCCOCCOCCOCCC(=O)NC(CS)C(N)=O]

In yet a particular embodiment, in formula (I-1-1), W is absent, Y is Q, Q is glutamine (Gln), n=3, Lk is L₁-L₂-L₃, L₁ is —NH—, L₃ is —(CO)—, L₂ is —(C₂H₄—O)ᵢ—C₂H₄—, i=4, x is NH₂, and the structure of the linking unit is as follows (linking unit LU108):

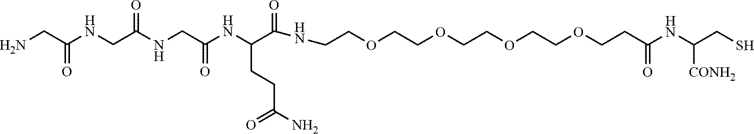 [SMILES: NCC(=O)NCC(=O)NCC(=O)NC(CCC(N)=O)C(=O)NCCOCCOCCOCCOCCC(=O)NC(CS)C(N)=O]

In a particular embodiment, in formula (I-1-1), both Y and W are absent, n=3, Lk is L₁-L₂-L₃, L₁ is —NH—, L₃ is —(CO)—, L₂ is —C₅H₁₀—, and the structure of the linking unit is as follows (linking unit LU109):

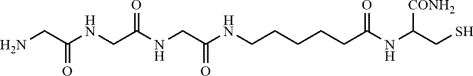 [SMILES: NCC(=O)NCC(=O)NCC(=O)NCCCCCC(=O)NC(CS)C(N)=O]

In yet a particular embodiment, in formula (I-1-1), both Y and W are absent, n=3, Lk is L₁-L₂-L₃, L₁ is —NH—, L₃ is —(CO)—, L₂ is —C₅H₁₀— group substituted with one —NR₁R₂ group, R₁ is hydrogen, R₂ is —(CO)CH₃, x is NH₂, and the structure of the linking unit is as follows (linking unit LU110):

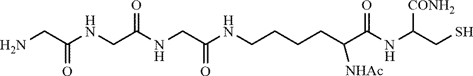 [SMILES: CC(=O)NC(CCCCNC(=O)CNC(=O)CNC(=O)CN)C(=O)NC(CS)C(N)=O]

The linking unit of formula (I-2), when D2 is LPXTG and A1 is

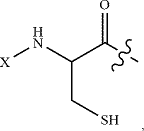

the structure of the compound of formula (I-2) is as shown in the following formula (I-2-1):

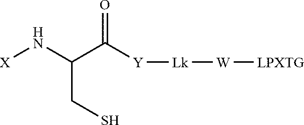

wherein x is selected from hydrogen, an amino acid fragment comprising 1-10 amino acids, a nucleotide fragment comprising1-10 nucleotides;
Y, Lk and W are as defined in formula (I), respectively.

In one embodiment, x is hydrogen.

### Compound of Formula (I) as Linking Unit

In one embodiment, the reactive group comprised by A1 or A2 can be used to covalently couple with a payload containing another reactive group, such that the compound of formula (I) bears a payload.

In another embodiment, the ligase recognition sequence comprised by D1 or D2 can be used in the coupling by a ligase with the corresponding ligase recognition sequence. As the result, a compound of formula (I) can be linked to a molecule comprising a ligase recognition sequence, wherein the ligase recognition sequence comprised by the said molecule is a ligase donor/acceptor substrate recognition sequence corresponding to the ligase recognition sequence comprised by D1 or D2.

In one embodiment, the molecule comprises a recognition sequence of the ligase donor substrate, and correspondingly, D1 or D2 is independently a recognition sequence of the ligase acceptor substrate. In another embodiment, the molecule comprises a recognition sequence of the ligase acceptor substrate, and correspondingly, D1 or D2 is independently a recognition sequence of the ligase donor substrate.

Thus, a compound of formula (I) can be used as a linking unit that can be linked to a targeting molecule (such as an antibody or antigen-binding fragment thereof) and/or a payload. The linking unit may contain a ligase recognition sequence for coupling of the linking unit with the targeting molecule. The linking unit may also contain a reactive group for covalent coupling with the payload.

Depending on the type of terminal modification of the targeting molecule to be coupled, the ligase recognition sequence comprised by the linking unit is a recognition sequence of the ligase acceptor substrate or a recognition sequence of the ligase donor substrate. The recognition sequences correspond to the ligase employed.

Depending on the type of reactive group of the payload to be coupled, the reactive group comprised by the linking unit belongs to the type that can undergo condensation reaction therewith.

The linking unit may influence the properties of the drug conjugate formed thereby. For example, the linking unit can optionally be used to provide suitable hydrophilicity, and can optionally contain cleavage site(s) to achieve a suitable release profile of the payload.

In an alternative embodiment, the linking unit further comprises one or more non-enzymatic cleavage sites, each independently located at any suitable position. In one embodiment, the non-enzymatic cleavage site may be a hydrazone that is sensitive to pH. In another embodiment, the non-enzymatic cleavage site is a disulfide bond that is sensitive to reducing agents. In another alternative embodiment, the linking unit further comprises one or more enzymatic cleavage sites, each independently located at any suitable position beyond Y and W. In one embodiment, the enzymatic cleavage site is selected from the group consisting of a oligomeric peptide that is sensitive to protease, a cathepsin cleavage site, a glutaminase cleavage site, and the combination thereof.

In yet another alternative embodiment, to increase the payloading of the targeting molecule-drug conjugate, the linking unit may further comprise a branched structural fragment. The backbone of this branched structure is formed by multifunctional molecules according to a particular linking pattern, and the number and structure of the branches can be made to accommodate the desired number of payloads. Each of the branches may comprise the structure of the linear linking unit described above.

One skilled in the art can synthesize the linking units by conventional solid phase or liquid phase methods for polypeptide synthesis.

### Payload-Bearing Formula (I) Compound

The reactive group comprised by A1 or A2 is covalently coupled with a payload containing another reactive group to give a payload-bearing formula (I) compound.

In yet another aspect, provided is a compound having the structure of formula (II)

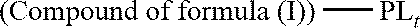

wherein
PL is a Payload which is linked to the A1 or A2 moiety of the compound of formula (I);
t is an integer of 1 to 20.
t denotes the number of PLs coupled with the compound of formula (I).

In one embodiment, t is an integer of 1 to 10; e.g., 1, 2, 3, 4, 5, 6, 7, 8, 9, or 10.

In one embodiment, t is 1 and the compound of formula (II) has the structure of the following formula (II-1) or formula (II-2):

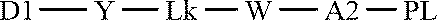

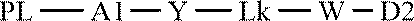

wherein, A1, A2, D1, D2, Y, Lk, and W are as defined above, respectively.

In another embodiment, t is 2-20, the structure of the compound of formula (II) is as shown in any one of the following formula (II-3) to formula (II-6):

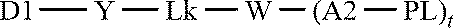

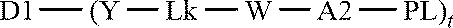

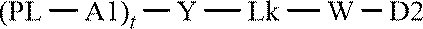

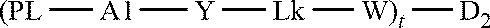

wherein, A1, A2, D1, D2, Y, Lk, and W are as defined in formula (II-1) or formula (II-2), respectively.

### Payload

In the present disclosure, the payload may be selected from the group consisting of small molecule compounds, nucleic acids and analogues, tracer molecules (including fluorescent molecules, etc.), short peptides, polypeptides, peptidomimetics, and proteins. In one embodiment, the payload is selected from the group consisting of small molecule compounds, nucleic acid molecules, and tracer molecules. In a preferred embodiment, the payload is selected from small molecule compounds. In a more preferred embodiment, the payload is selected from the group consisting of cytotoxin and fragments thereof.

In one embodiment, the cytotoxin is selected from the group consisting of drugs that target microtubule cytoskeleton.

In a preferred embodiment, the cytotoxin is selected from the group consisting of taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhy-drovinblastine, dolastatin 10 and analogues, halichondrin B and eribulin, indole-3-oxoacetamide, podophyllotoxins, 7-diethylamino-3-(2′-benzoxazolyl)-coumarin (DBC), discodermolide, laulimalide.

In another embodiment, the cytotoxin is selected from the group consisting of DNA topoisomerase inhibitors such as camptothecins and derivatives thereof, mitoxantrone, mitoguazone.

In a preferred embodiment, the cytotoxin is selected from the group consisting of nitrogen mustards such as chlorambucil, chlornaphazine, cholophosphamide, estramustine, ifosfamide, mechlorethamine, mechlorethamine oxide hydrochloride, melphalan, novembichin, phenamet, phenesterine, prednimustine, trofosfamide, uracil mustard.

In yet another preferred embodiment, the cytotoxin is selected from the group consisting of nitrosoureas such as carmustine, flubenzuron, formoterol, lomustine, nimustine, ramustine.

In one embodiment, the cytotoxin is selected from the group consisting of aziridines.

In a preferred embodiment, the cytotoxin is selected from the group consisting of benzodopa, carboquone, meturedepa, and uredepa.

In one embodiment, the cytotoxin is selected from the group consisting of an anti-tumor antibiotic.

In a preferred embodiment, the cytotoxin is selected from the group consisting of enediyne antibiotics.

In a more preferred embodiment, the cytotoxin is selected from the group consisting of dynemicin, esperamicin, neocarzinostatin, and aclacinomycin.

In another preferred embodiment, the cytotoxin is selected from the group consisting of actinomycin, antramycin, bleomycins, actinomycin C, carabicin, carminomycin, and cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin.

In yet another preferred embodiment, the cytotoxin is selected from the group consisting of trichothecene.

In a more preferred embodiment, the cytotoxin is selected from the group consisting of T-2 toxin, verracurin A, bacillocporin A, and anguidine.

In one embodiment, the cytotoxin is selected from the group consisting of an anti-tumor amino acid derivatives.

In a preferred embodiment, the cytotoxin is selected from the group consisting of ubenimex, azaserine, 6-diazo-5-oxo-L-norleucine.

In another embodiment, the cytotoxin is selected from the group consisting of folic acid analogues.

In a preferred embodiment, the cytotoxin is selected from the group consisting of dimethyl folic acid, methotrexate, pteropterin, trimetrexate, and edatrexate.

In one embodiment, the cytotoxin is selected from the group consisting of purine analogues.

In a preferred embodiment, the cytotoxin is selected from the group consisting of fludarabine, 6-mercaptopurine, tiamiprine, thioguanine.

In yet another embodiment, the cytotoxin is selected from pyrimidine analogues.

In a preferred embodiment, the cytotoxin is selected from the group consisting of ancitabine, gemcitabine, enocitabine, azacitidine, 6-azauridine, carmofur, cytarabine, dideoxyuridine, doxifluridine, floxuridine.

In one embodiment, the cytotoxin is selected from the group consisting of androgens.

In a preferred embodiment, the cytotoxin is selected from the group consisting of calusterone, dromostanolone propionate, epitiostanol, mepitiostane, testolactone.

In another embodiment, the cytotoxin is selected from the group consisting of anti-adrenals.

In a preferred embodiment, the cytotoxin is selected from the group consisting of aminoglutethimide, mitotane, and trilostane.

In one embodiment, the cytotoxin is selected from the group consisting of anti-androgens.

In a preferred embodiment, the cytotoxin is selected from the group consisting of flutamide, nilutamide, bicalutamide, leuprorelin acetate, and goserelin.

In yet another embodiment, the cytotoxin is selected from the group consisting of a protein kinase inhibitor and a proteasome inhibitor.

In another embodiment, the cytotoxin is selected from the group consisting of vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, and pyrrolobenzodiazepine (PBD).

In a particularly embodiment, the cytotoxin is selected from the group consisting of vinblastines, colchicines, taxanes, auristatins, and maytansinoids.

In another particular embodiment, the cytotoxin is an maytansinoid, such as DM1 and the like. Note that where a cytotoxin comprising a thiol moiety is used, the thiol moiety being capable of reaction with a maleimide moiety to form a thiosuccinimide, for example a maytansinoid, e.g., DM1, the cytotoxin can link directly via the thiosuccinimide. In such case, it could be understood that in some embodiments Payload and the thiol moiety together constitutes a cytotoxin, and therefore in such case Payload represents the rest moiety of the cytotoxin molecule except for the thiol moiety.

In a particular embodiment, the cytotoxin is an auristatin, such as MMAE (monomethyl auristatin E), MMAF (monomethyl auristatin F), MMAD (monomethyl auristatin D) and the like. The synthesis and structure of austenitic compounds are described in US20060229253, the entire disclosure of which is incorporated herein by reference.

The payload contains a reactive group which can react with the reactive group in the compound of formula (I) and thus covalently couple the payload with the compound of formula (I). Compounds that do not contain reactive groups require appropriate derivatization to give the payload. In one embodiment, the reactive group in the payload is maleimide, and the compound without maleimide may be subjected to suitable reaction(s) to give a maleimide derivative. For example MMAF is derivatized to give mc-MMAF (mc is maleimidocaproyl). MMAE is derivatized to give mc-Val-Cit-PAB-MMAE. mc in the above structures can be replaced by mcc (4-(maleimidomethyl)cyclohexane-1-carbonyl) or maleimide-R structure, wherein R is a C₁₋₂₀ alkylene, and optionally one or more (-CH₂-) structures in the alkylene can be replaced by -O-.

In one embodiment, A1 or A2 in the compound of formula (I) are each independently cysteine with an optional derivatization. The amino group in the cysteine structure is connected to the rest of the compound of formula (I), and the thiol group in the cysteine structure reacts with a maleimide group in a payload which contains a maleimide or maleimide derivative structure, resulting in a payload-bearing formula (I) compound, which comprises a thiosuccinimide structure.

In a particular embodiment, the thiol group in the cysteine structure in the compound of formula (I) is connected to the maleimide or maleimide derivative in the payload by Michael addition.

Thiosuccinimide is unstable under physiological conditions and is liable to reverse Michael addition which leads to cleavage at the coupling site. Moreover, when another thiol compound is present in the system, thiosuccinimide may also undergo thiol exchange with the other thiol compound. Both of these reactions cause the fall-off of the payload and result in toxic side effects. In the present disclosure, the ring opening of the succinimide is conducted using a ring opening reaction after the step of Michael addition. After ring opening, the succinimide no longer undergoes reverse Michael addition or thiol exchange, and thus the product is more stable. Method of ring opening reaction can be found in WO2015165413A1.

The ring-opened compound of formula (I) can be purified by semi-preparative/preparative HPLC or other suitable separation means to obtain payload-bearing formula (I) compound with high purity and defined composition, regardless of the efficiency of the succinimide ring opening reaction.

### Specific Embodiment of the Payload-Bearing Formula (I) Compound

In yet another aspect, provided is also a compound of the following formulas (i), (ii), (iii), (iii’), (iv), (v), (v′), (vi), (vii) or (vii’).

In some embodiments of the present disclosure, when D1 is Gₙ, G is glycine, and A2

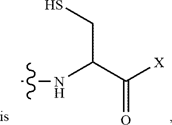

the structure of the compound of formula (II-1) is as shown in formula (i):

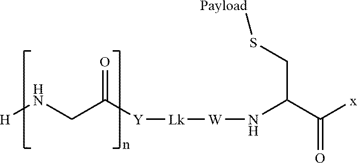

where n is an integer of 3 to 10;
x is selected from the group consisting of hydrogen, OH, NH₂, an amino acid fragment comprising 1-10 amino acids, a nucleotide fragment comprising 1-10 nucleotides;
payload, Y, W, and Lk are as defined in formula (II), respectively.

In a preferred embodiment, x is OH, NH₂ or Gly.

In another embodiment, in formula (i), Y and W are both absent, the payload is a mc-toxin formed by derivatization of a cytotoxin, wherein toxin represents a cytotoxin, and the structure of the compound of formula (i) is as shown in the following formula (ii):

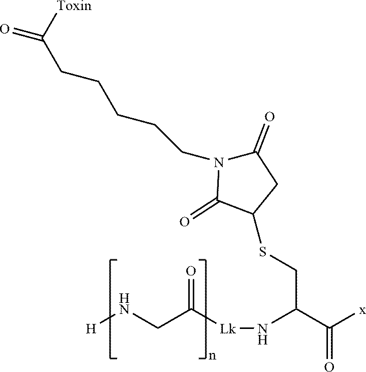

wherein n, Lk and x are as defined in formula (i), respectively.

The mc structure can be further subjected to ring opening reaction according to a known method to obtain compounds of formula (iii) and formula (iii′).

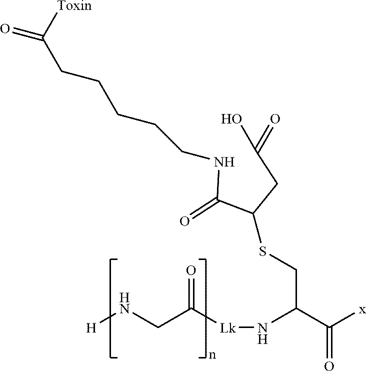

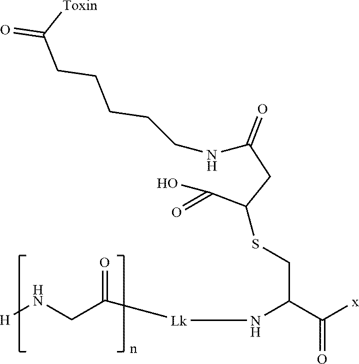

Formula (iii) and formula (iii’) are isomers, wherein n, Lk, and x are as defined in formula (i), respectively.

In a preferred embodiment, the cytotoxin in formula (ii) is MMAF, i.e., the payload is mc-MMAF, and the structure of the compound of formula (ii) is as shown in the following formula (iv):

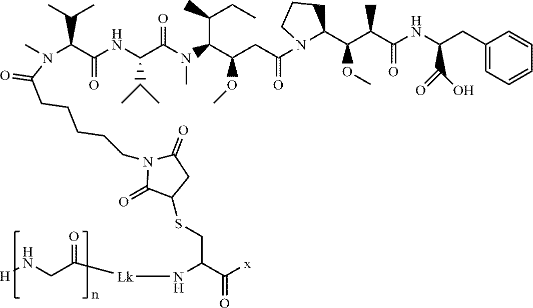

wherein n, Lk and x are as defined in formula (i), respectively.

The mc structure can be further subjected to ring opening reaction according to a known method to obtain compounds of formula (v) and formula (v′).

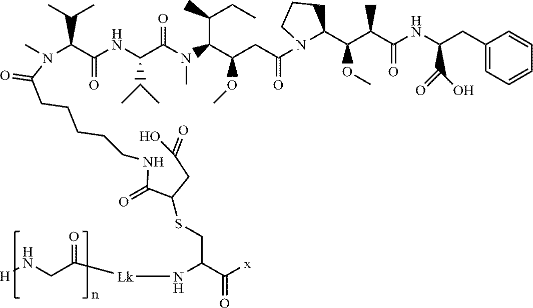

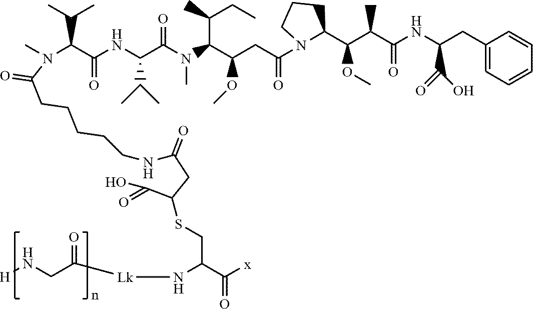

Formulae (v) and (V′) are isomers, wherein n, Lk and x are as defined in formula (i), respectively.

In a more preferred embodiment, in formula (iv), n=3, Lk is L₁-L₂-L₃, L₁ is —NH—, L₃ is —(CO)—, L₂ is —(C₂H₄—O)ᵢ—C₂H₄—, i=4, x is NH₂, and the structure of the compound of formula (iv) is as shown in formula (vi) (IM102 (ring closed)):

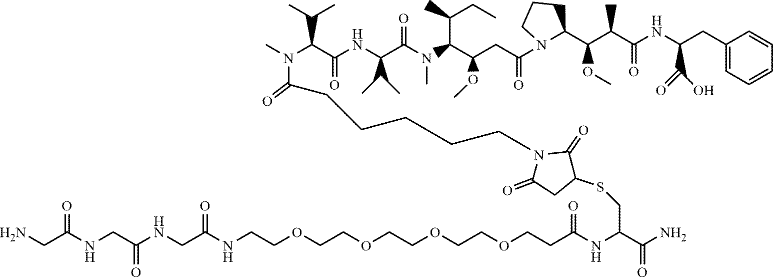 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@H](NC(=O)[C@H](C(C)C)N(C)C(=O)CCCCCN1C(=O)CC(SCC(NC(=O)CCOCCOCCOCCOCCNC(=O)CNC(=O)CNC(=O)CN)C(N)=O)C1=O)C(C)C]

The mc structure can be further subjected to ring opening reaction according to a known method to obtain compounds of formula (vii) and formula (vii′) (IM102 (ring open)).

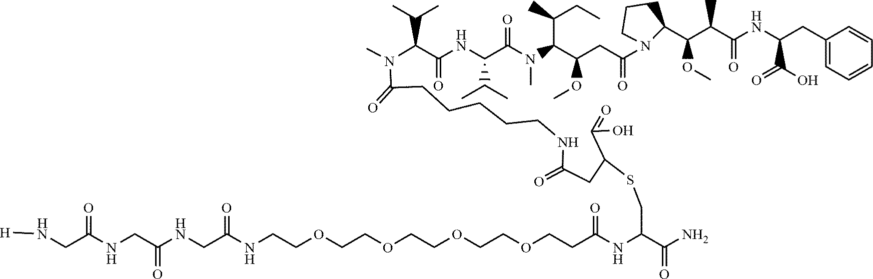 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCNC(=O)CC(SCC(NC(=O)CCOCCOCCOCCOCCNC(=O)CNC(=O)CNC(=O)CN)C(N)=O)C(=O)O)C(C)C]

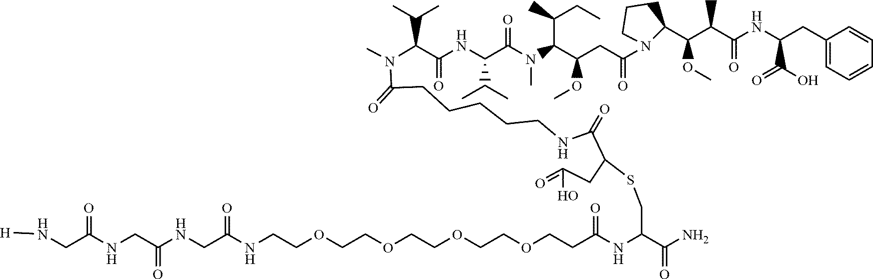 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCNC(=O)C(CC(=O)O)SCC(NC(=O)CCOCCOCCOCCOCCNC(=O)CNC(=O)CNC(=O)CN)C(N)=O)C(C)C]

Formulae (vii) and (vii’) are isomers.

### Conjugates and Preparation Thereof

Furthermore, the payload-bearing formula (I) compound which has the moiety comprising ligase recognition sequence can be coupled with other molecules comprising a ligase recognition sequence, and can be thereby used in for example, the preparation of a targeting molecule-drug conjugate, such as an antibody-drug conjugate. Accordingly, in yet another aspect, provided is a conjugate which comprises a compound of formula (I), a targeting molecule, and a payload.

### Specific Constitution of the Conjugate

In yet another aspect, provided is a conjugate having the structure of formula (III)

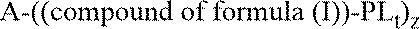

wherein
PL is a payload which is linked to the A1 or A2 moiety of the compound of formula (I);
A is a targeting molecule which is linked to the D1 or D2 moiety of the compound of formula (I);
z is an integer of 1 to 20;
t is an integer of 1 to 20.
t represents the number of PL(s) linked to the compound of formula (I).

In one embodiment, the ligase recognition sequence represented by D1 or D2 in the compound of formula (I) corresponds to the ligase recognition sequence in the targeting molecule which is to be coupled therewith, and site-specific coupling of the targeting molecule with the compound of formula (I) is thus realized. When the terminal modification of the targeting molecule to be coupled is a terminal modification based on a recognition sequence of the ligase donor substrate, D1 or D2 is independently a recognition sequence of the ligase acceptor substrate. Alternatively, when the terminal modification of the targeting molecule to be coupled is a terminal modification based on a recognition sequence of the ligase acceptor substrate, D1 or D2 is independently a recognition sequence of the ligase donor substrate.

In one embodiment, z is an integer of 1 to 10; e.g., 1, 2, 3, 4, 5, 6, 7, 8, 9, or 10.

In one embodiment, t is an integer of 1 to 10; e.g., 1, 2, 3, 4, 5, 6, 7, 8, 9, or 10.

In one embodiment, t is 1, conjugate of formula (III) has the structure of the following formula (III-1) or formula (III-2):

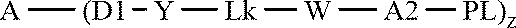

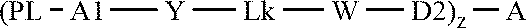

wherein, PL, A1, A2, D1, D2, Y, W, Lk, and z are as defined above, respectively.

In another embodiment, t is 2-20, conjugate of formula (III) has the structure of any of the following formulas (III-3) to (III-6):

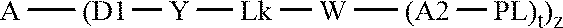

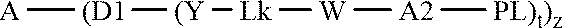

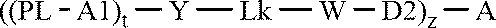

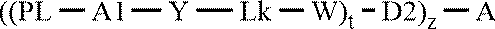

wherein, PL, A1, A2, D1, D2, Y, W, Lk, and z are as defined in formula (III-1) or formula (III-2), respectively.

### Targeting Molecule

In one embodiment, the targeting molecule is an antibody or an antigen binding fragment thereof.

In some embodiments of the present disclosure, targets recognized by the targeting molecules (such as antibodies or antigen-binding fragments thereof) include but are not limited to CD19, CD22, CD25, CD30/TNFRSF8, CD33, CD37, CD44v6, CD56, CD70, CD71, CD74, CD79b, CD117/KIT, CD123, CD138, CD142, CD174, CD227/MUC1, CD352, CLDN18.2, DLL3, ErbB2/HER2, CN33, GPNMB, ENPP3, Nectin-4, EGFRvIII, SLC44A4/AGS-5, mesothelin, CEACAM5, PSMA, TIM1, LY6E, LIV1, Nectin4, SLITRK6, HGFR/cMet, SLAMF7/CS1, EGFR, BCMA, AXL, NaPi2B, GCC, STEAP1, MUC16, Mesothelin, ETBR, EphA2, 5T4, FOLR1, LAMP1, Cadherin 6, FGFR2, FGFR3, CA6, CanAg, Integrin αV, TDGF1, Ephrin A4, Trop2, PTK7, NOTCH3, C4.4A, FLT3.

In one embodiment, the targeting molecule is an anti-human HER2 antibody or antigen binding fragment thereof. Examples of anti-human HER2 antibodies include but are not limited to Pertuzumab and Trastuzumab. Pertuzumab binds to the second extracellular domain (ECD2) of HER2 and is approved for the treatment of HER2-positive breast cancer. Trastuzumab binds to the fourth extracellular domain (ECD4) of HER2 and is approved for the treatment of Her2-positive breast cancer and gastric cancer.

In a preferred embodiment, the anti-human TROP2 antibody is one or more selected from engineered anti-HER2 antibodies based on Pertuzumab (Perjeta®, Genentech).

In one embodiment, the targeting molecule is one or more selected from anti-human TROP2 antibodies or antigen-binding fragment thereof. In a particular embodiment, the anti-human TROP2 antibody is one or more selected from engineered anti-TROP2 antibodies based on hRS7 (US20140120035). In another particular embodiment, the anti-human TROP2 antibody is one or more selected from engineered anti-TROP2 antibodies based on MAAA1181a (US20160297890). In yet another particular embodiment, the anti-human TROP2 antibody is one or more selected from optionally engineered anti-TROP2 antibodies based on Ab0052, Ab0053, Ab0054, Ab0061, Ab0062, Ab0063 or Ab0064.

In a preferred embodiment, the anti-human HER2 or TROP2 antibody is a recombinant antibody selected from the group consisting of monoclonal antibody, chimeric antibody, humanized antibody, antibody fragment, and antibody mimic. In one embodiment, the antibody mimic is selected from the group consisting of scFv, minibody, diabody, nanobody. For the coupling with the compound of formula (I), the targeting molecule of the present disclosure may comprise a modified moiety to connect with D1 or D2 in the compound of formula (I). The introduction position of such modified moiety is not limited, for example, when the targeting molecule is an antibody, its introduction position can be, but not limited to, located at the C-terminal or the N-terminal of the heavy chain or light chain of the antibody.

In an alternative embodiment, a modified moiety for the coupling with D1 or D2 in the compound of formula (I) can be introduced at a non-terminal position of the heavy chain or light chain of the antibody using, for example, chemical modification methods.

In one embodiment, the targeting molecule of the present disclosure is an antibody or antigen-binding fragment thereof, which may comprise terminal modification. A terminal modification refers to a modification at the C-terminal or N-terminal of the heavy chain or light chain of the antibody, which for example comprises a ligase recognition sequence. In another embodiment, the terminal modification may further comprise spacer Sp2 comprising 2-100 amino acids, wherein the antibody, Sp2 and the ligase recognition sequence are sequentially linked. In a preferred embodiment, Sp2 is a spacer sequence containing 2-20 amino acids. In a particular embodiment, Sp2 is a spacer sequence selected from the group consisting of GA, GGGS and GGGGSGGGGS, especially GA.

In a preferred embodiment, the light chain of the antibody or antigen-binding fragment thereof includes 3 types: wild-type (LC); the C-terminus modified light chain (LCCT), which is modified by direct introduction of an ligase recognition sequence LPXTG and C-terminus modified light chain (LCCT_{L}), which is modified by introduction of short peptide spacers plus the ligase donor substrate recognition sequence LPXTG. The heavy chain of the antibody or antigen-binding fragment thereof includes 3 types: wild-type (HC); the C-terminus modified heavy chain (HCCT), which is modified by direct introduction of an ligase recognition sequence LPXTG; and C-terminus modified heavy chain (HCCT_{L}), which is modified by introduction of short peptide spacers plus the ligase donor substrate recognition sequence LPXTG. X can be any natural or non-natural single amino acid. When z in the compound of formula (III) is 1 or 2, the combination of the above heavy and light chains can form 8 preferred antibody molecules, see the amino acid sequence table.

In a preferred embodiment, the light chain of the antibody or antigen-binding fragment thereof includes 3 types: wild-type (LC); the N-terminus modified light chain (LCNT), which is modified by direct introduction of an ligase recognition sequence GGG; and N-terminus modified light chain (LCNT_{L}), which is modified by introduction of short peptide spacers plus the ligase acceptor substrate recognition sequence GGG. The heavy chain of the antibody or antigen-binding fragment thereof includes 3 types: wild-type (HC); the N-terminus modified heavy chain (HCNT), which is modified by direct introduction of an ligase recognition sequence GGG; and N-terminus modified heavy chain (HCNT_{L}), which is modified by introduction of short peptide spacers plus the ligase acceptor substrate recognition sequence GGG.

In a particular embodiment, the targeting molecule is an antibody, which comprises a light chain and a heavy chain having the amino acid sequences of SEQ ID No. 1 and SEQ ID No. 2 (P-LCCT_{L}-HC); SEQ ID No. 3 and SEQ ID No. 4 (P-LC-HCCT); SEQ ID No. 5 and SEQ ID No. 6 (P-LC-HCCT_{L}); SEQ ID No. 7 and SEQ ID No. 8 (P-LCCT-HC); SEQ ID No. 9 and SEQ ID No. 10 (P-LCCT-HCCT); SEQ ID No. 11 and SEQ ID No. 12 (P-LCCT-HCCT_{L}); SEQ ID No. 13 and SEQ ID No. 14 (P-LCCT_{L}-HCCT); SEQ ID No. 15 and SEQ ID No. 16 (P-LCCT_{L}-HCCT_{L}); SEQ ID No. 17 and SEQ ID No. 18 (modified hRS7); SEQ ID No. 19 and SEQ ID No. 20 (modified MAAA1181a); SEQ ID No. 21 and SEQ ID No. 22 (Ab0052); SEQ ID No. 23 and SEQ ID No. 24 (Ab0053); SEQ ID No. 25 and SEQ ID No. 26 (Ab0054); SEQ ID No. 27 and SEQ ID No. 28 (Ab0061); SEQ ID No. 29 and SEQ ID No. 30 (Ab0062); SEQ ID No. 31 and SEQ ID No. 32 (Ab0063); or SEQ ID No. 33 and SEQ ID No. 34 (Ab0064), respectively.

The conjugates of the present disclosure can further comprise a payload. The payload is as described above.

### Specific Embodiments for the Conjugate

On the other hand, provided is a conjugate of the following formulas (1), (2), (2′), (3), (3′), (4), (4’).

A conjugate of formula (III-1), wherein when D1 is Gₙ, G is glycine, and A2 is

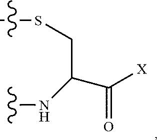

which is the remaining residue of

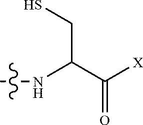

after the reaction of the thiol group with a payload, and the structure of the conjugate is as shown in the following formula (1):

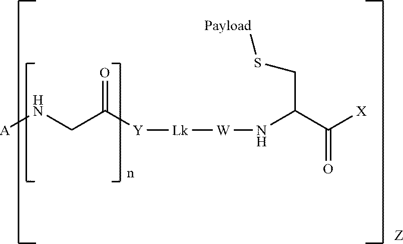

wherein A is a targeting molecule;
n is an integer of 3 to 10;
x is OH, NH₂ or Gly;
Payload, Y, Lk, W, and z are as defined in formula (III-1), respectively.

In a preferred embodiment, Y and W are both absent, the payload in formula (1) is mc(ring open)-toxin, toxin represents a cytotoxin, and the structure of the conjugate is as shown in the following formula (2) or formula (2’):

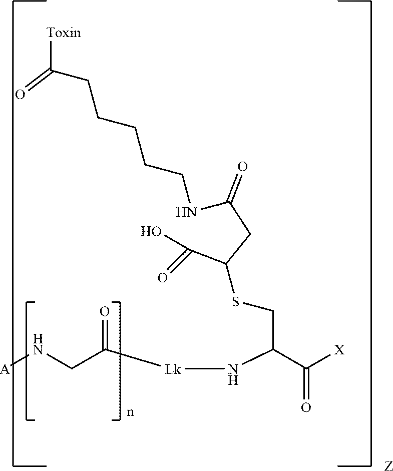

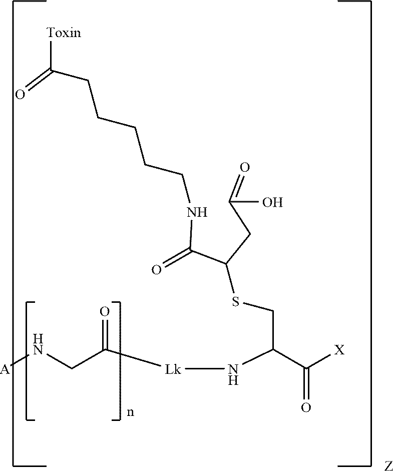

wherein A, toxin, n, Lk, x and z are as defined in formula (1), respectively.

In a more preferred embodiment, the cytotoxin in formula (2) and formula (2′) is MMAF, i.e., the payload is mc(ring open)-MMAF, and the structure of the conjugate is as shown in the following formula (3) or formula (3′):

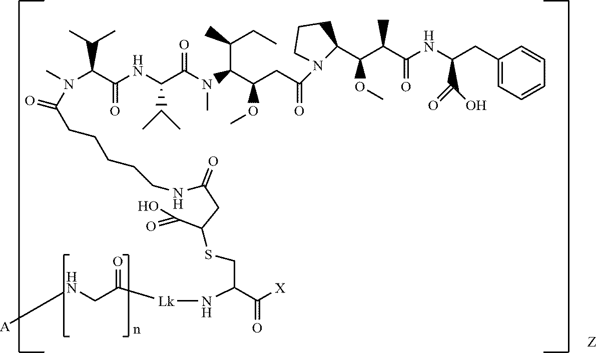

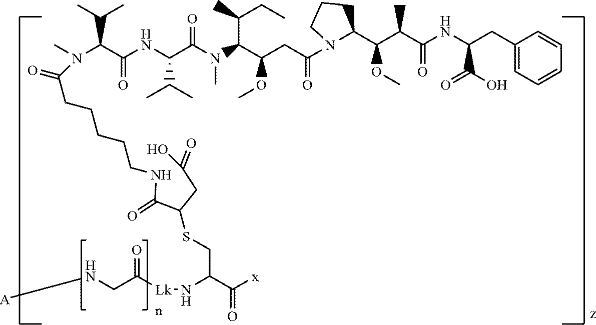

Formulae (3) and (3’) are isomers, wherein:
A, n, Lk and x are as defined in formula (1), respectively;
z is any integer of 1 to 20, for example z can be 1, 2, 3, 4, 5, 6, 7, 8, 9, 10, 11, 12, 13, 14, 15, 16, 17, 18, 19 or 20. In one embodiment, z is selected from the following values: an integer of 1 to 10, 1 to 8, 1 to 6 or 1 to 4. In another embodiment, z is 1 or 2. In a very special embodiment, z is 2.

In a particular embodiment, the targeting molecule is the antibody Pertuzumab, hRS7, MAAA1181a, Ab0052, Ab0053, Ab0054, Ab0061, Ab0062, Ab0063 or Ab0064.

In another specific embodiment, n=3, Lk is L₁-L₂-L₃, L₁ is —NH—, and L₃ is —(CO)—, L₂ is —(C₂H₄—O)ᵢ—C₂H₄—, i=4, x is NH₂, z=2. The structure of the conjugate is as shown in the following formula (4) and (4′):

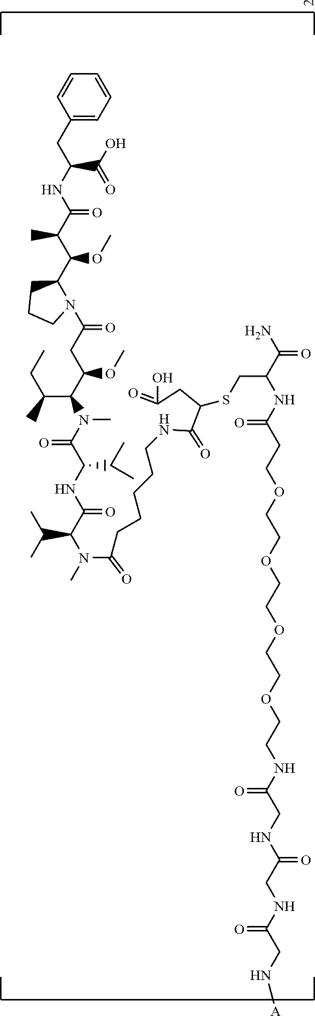

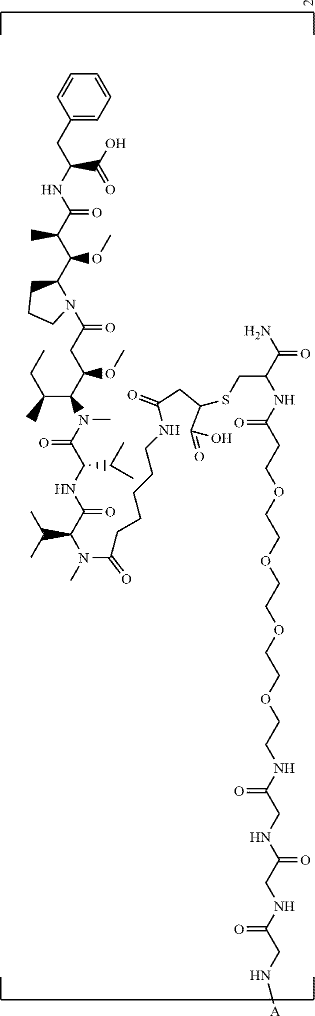

Formulae (4) and (4’)are isomers, wherein A is a modified Pertuzumab, a modified hRS7 or a modified MAAA1181a, or is Ab0052, Ab0053, Ab0054, Ab0061, Ab0062, Ab0063 or Ab0064.

### Preparation of the Conjugate

The conjugates of the present disclosure can be prepared by any method known in the art. In some embodiments, the conjugate is prepared by the ligase-catalyzed site-specific coupling of a targeting molecule and a payload-bearing formula (I) compound, wherein the targeting molecule is modified by a ligase recognition sequence. The method comprises step A and step B.

### Step A. Preparation of the Linking Unit-Payload Intermediate

In a preferred embodiment, A1 or A2 in the compound of formula (I) is each independently covalently linked via a reactive group to a payload containing another reactive group. In a preferred embodiment, A1 or A2 in the compound of formula (I) is each independently covalently linked to the maleimide group in the payload which contains a maleimide structure or derivative thereof, through a disulfide bond, a thioether bond, a thioester bond, or an urethane bond, giving a linking unit-payload intermediate, i.e., the compound of formula (I) with a payload.

The linking unit-payload intermediate prepared using the compound of formula (I) of the present disclosure has defined structure, defined composition and high purity, so that when the coupling reaction with an antibody is conducted, fewer impurities are introduced or no other impurities are introduced. When such an intermediate is used for the ligase-catalyzed site-specific conjugation with a modified antibody containing a ligase recognition sequence, a homogeneous ADC with highly controllable quality is obtained.

### Step B. Linking the Targeting Molecule to the Payload-Bearing Formula (I) Compound

The targeting molecule of the present disclosure can be coupled with the payload-bearing formula (I) compound (i.e., the compound of formula (II)) by any method known in the art. For example, ligase-catalyzed site-specific coupling technique is applied, and the targeting molecule and the payload-bearing formula (I) compound are linked to each other via the ligase-specific recognition sequences of the substrates. The recognition sequence depends on the particular ligase employed. In one embodiment, the targeting molecule is an antibody with recognition sequence-based terminal modifications introduced at the C-terminal of the light chain and/or the heavy chain, and the targeting molecule is coupled with the compound of formula (II), under the catalysis of the wild type or optimized engineered ligase or any combination thereof, and under suitable catalytic reaction conditions.

In a specific embodiment, the ligase is Sortase A and the coupling reaction can be represented by the following scheme:

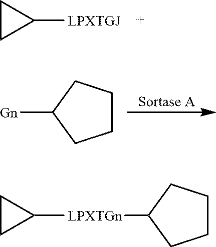

The triangle and pentagon respectively represent any of the following: a portion of an antibody or a portion of a compound of formula (II), and the positions being interchangeable. n, X and J are respectively as defined above. When coupled with Gₙ, which is the corresponding recognition sequence of the acceptor substrate, the upstream peptide bond of the glycine in the LPXTGJ sequence is cleaved by Sortase A, and the resulting intermediate is linked to the free N-terminal of Gₙ to generate a new peptide bond. The resulting amino acid sequence is LPXTGₙ. The sequences Gₙ and LPXTGJ are as defined above.

### Pharmaceutical Composition and Pharmaceutical Preparation

Another object of the disclosure is to provide a pharmaceutical composition comprising a prophylactically or therapeutically effective amount of a conjugate of the present disclosure, and at least one pharmaceutically acceptable carrier.

The pharmaceutical composition of the present disclosure may be administered in any manner as long as it achieves the effect of preventing, alleviating, preventing or curing the symptoms of a human or animal. For example, various suitable dosage forms can be prepared according to the administration route, especially injections such as lyophilized powder for injection, injection, or sterile powder for injection.

The term “pharmaceutically acceptable” means that when contacted with tissues of the patient within the scope of normal medical judgment, no undue toxicity, irritation or allergic reaction, etc. shall arise, having reasonable advantage-disadvantage ratios and effective for the intended use.

The term pharmaceutically acceptable carrier refers to those carrier materials which are pharmaceutically acceptable and which do not interfere with the bioactivities and properties of the conjugate. Examples of aqueous carriers include but are not limited to buffered saline, and the like. The pharmaceutically acceptable carrier also includes carrier materials which brings the composition close to physiological conditions, such as pH adjusting agents, buffering agents, toxicity adjusting agents and the like, and sodium acetate, sodium chloride, potassium chloride, calcium chloride, sodium lactate, and the like.

In one embodiment, the pharmaceutical composition of the present disclosure has a drug to antibody ratio (DAR) of an integer or non-integer of 1 to 20, such as 1-10, 1-8, 1-6, 1-4, 1-3, 1-2.5, 1-2, 1-1.5, 1.5-2 or 1.5-2.5. In a particular embodiment, the conjugate of the present disclosure has a DAR of 1.6-2.1. In another particular embodiment, the DAR of the conjugate of the disclosure is 1.8-1.9.

### Treatment Method and Use

The conjugates of the present disclosure are useful for the treatment of tumors and/or autoimmune diseases. Tumors susceptible to conjugate treatment include those characterized by specific tumor-associated antigens or cell surface receptors, and those will be recognized by the targeting molecule in the conjugate and can be killed by the payload/cytotoxin in the conjugate.

Accordingly, in yet another aspect, also provided is use of a conjugate of the present disclosure or a pharmaceutical composition of the present disclosure in the manufacture of a medicament for treating a disease, disorder or condition selected from a tumor or an autoimmune disease.

In another aspect, provided is a conjugate of the present disclosure or a pharmaceutical composition of the present disclosure for use in the treatment of a tumor or an autoimmune disease.

In a further aspect, provided is a method of treating a tumor or an autoimmune disease, the method comprising administering to an individual in need thereof an effective amount of a conjugate of the present disclosure or a pharmaceutical composition of the present disclosure

In a preferred embodiment, the conjugate of the present disclosure formed by coupling of the anti-human HER2 antibody and the small molecule cytotoxin can specifically bind to HER2 on the surface of the tumor cell and selectively kill the HER2-expressing tumor cells. In another preferred embodiment, provided is use of a conjugate of the present disclosure or a pharmaceutical composition of the present disclosure in the manufacture of a medicament for treating a disease, disorder or condition selected from HER2-positive tumors. In a more preferred embodiment, the disease, disorder or condition is selected from the group consisting of breast cancer, gastric cancer, lung cancer, ovarian cancer, urothelial cancer, and the like.

In a preferred embodiment, the conjugate of the present disclosure formed by the coupling of the anti-human HER2 antibody and the small molecule cytotoxin can specifically bind to TROP2 on the surface of the tumor cell and selectively kill the TROP2-expressing tumor cells. In another preferred embodiment, provided is use of a conjugate of the present disclosure or a pharmaceutical composition of the present disclosure in the manufacture of a medicament for treating a disease, disorder or condition selected from a TROP2-positive tumors. In a more preferred embodiment, the disease, disorder or condition is selected from the group consisting of breast cancer, urothelial carcinoma, lung cancer, liver cancer, endometrial cancer, head and neck cancer, ovarian cancer, and the like.

The dosage of the conjugate administered to the subject can be adjusted to a considerable extent. The dosage can vary according to the particular route of administration and the needs of the subject, and can be subjected to the judgment of the health care professional.

### Beneficial Effect

The present disclosure utilizes a linker with unique structure and uses a ligase to catalyze the coupling of the targeting molecule and the payload. The conjugate of the present disclosure has good homogeneity, high activity and high selectivity. In particular, the intracellular metabolites show significantly reduced cell proliferation toxicities to the cells with low expression or no expression of target antigens. Furthermore, the toxicity of the linking unit-payload intermediate is much lower than that of the free payload, and thus the manufacture process of the drug is less detrimental, which is advantageous for industrial production.

The conjugate of the present disclosure achieves at least one of the following technical effects:
(1) High inhibitory activity against target cells, or strong killing effect on target cells.
(2) Good physicochemical properties (e.g., solubility, physical and/or chemical stability).
(3) Good pharmacokinetic properties (e.g., good stability in plasma, appropriate half-life and duration of action).
(4) Good safety (low toxicity on non-target normal cells or tissues, and/or fewer side effects, wider treatment window), etc.

### Examples

### Preparation Example

In order to more clearly illustrate the objects and technical solutions, the present disclosure is further described below with reference to specific examples. It is to be understood that the examples are not intended to limit the scope of the disclosure. The specific experimental methods which were not mentioned in the following examples were carried out according to conventional experimental method.

### Instruments, Materials and Reagents

Unless otherwise stated, the instruments and reagents used in the examples are commercially available. The reagents can be used directly without further purification.

MS: Thermo Fisher Q Exactive Plus, Water2795-Quattro micro triple quadrupole mass spectrometer

HPLC: Waters 2695, Agilent 1100, Agilent 1200

Semi-preparative HPLC: Lisure HP plus 50D

Flow Cytometry: CytoFLEX S

HIC-HPLC: Butyl-HIC; mobile phase A: 25 mM PB, 2M (NH₄)₂SO₄, pH 7.0; mobile phase B: 25 mM PB, pH 7.0; flow rate: 0.8 ml/min; acquisition time: 25 min; injection amount: 20 µg; column temperature: 25° C.; detection wavelength: 280 nm; sample chamber temperature: 8° C.

SEC-HPLC: column: TSK-gel G3000 SWXL, TOSOH 7.8 mm ID × 300 mm, 5 µm; mobile phase: 0.2 M KH₂PO₄, 0.25 M KCl, pH 6.2; flow rate: 0.5 ml/min; acquisition time: 30 min; injection volume: 50 µl; column temperature: 25° C.; detection wavelength; 280 nm; sample tray temperature: 8° C.

CHO was obtained from Thermo Fisher Scientific; pcDNA 3.3 was obtained from Life Technology; HEK293F was obtained from Prejin; PEIMAX transfection reagent was obtained from Polyscience; MabSelect Sure ProA was obtained from GE; Capto S ImpAct was obtained from GE; Rink-amide-MBHA-resin and dichloro resin were obtained from Nankai synthesis; HCC1954 was obtained from ATCC CAT# CRL-2338; SK-BR-3 was obtained from ATCC CAT# HTB-30; BT-474 was obtained from ATCC CAT# HTB-20; NCI-N87 cells (ATCC CAT# CRL-5822); MCF7 was obtained from ATCC CAT# HTB-22; MDA-MB-231 was obtained from ATCC CAT# HTB-26; MDA-MB-468 was obtained from ATCC CAT# HTB-132; antibody Perjeta® is prepared according to the known sequence; antibody hRS7 is prepared according to the sequence in patent US 20140120035 (the heavy chain and light chain sequences of said antibody hRS7 are obtained from WHO drug information, INN list 113, Vol.29, No.2, 2015); antibody MAAA1181a is prepared according to the sequence in US 20160297890; optimized recombinant enzyme Sortase A derived from Staphylococcusaureus is prepared in E.coli.

### Example 1 Construction of Antibody Expression Vector, Antibody Expression, Purification and Identification

### 1.1 Construction of Expression Vector for the Modified Anti-Human HER2 Antibody

The expression plasmids for antibody P-LCCT_{L}-HC (light chain SEQ ID NO: 1, heavy chain: SEQ ID NO: 2) were constructed as follows. The sequence of the antibody P-LCCT_{L}-HC: based on the amino acid sequence of Pertuzumab, and GALPETGG was introduced at the C-terminal of the light chain.

1) According to the published amino acid sequence of Pertuzumab, the amino acid sequence of the light chain signal peptide was introduced at the N-terminal of the light chain, and the amino acid sequence GALPETGG was introduced at the C-terminal of the light chain, wherein LPETGG is the recognition sequence of the ligase donor substrate, and GA is a spacer sequence. The amino acid sequence of the heavy chain signal peptide was introduced at the N-terminal of the heavy chain. The light chain amino acid sequence and the heavy chain amino acid sequence were then subjected to codon optimization, respectively. The host for the optimization was CHO. Nucleotide sequences of the light chain and the heavy chain were obtained.

2) The recombinant arm sequence of the antibody expression vector pcDNA3.3 and the Kozak sequence (GCCGCCACC) were introduced at the 5’ end of the nucleotide sequence of the light chain obtained in 1) to give the expression vector for the light chain, and the recombination arm sequence of the antibody expression vector pcDNA3.3 and the Kozak sequence were introduced at the 5’ end of the nucleotide sequence of the heavy chain obtained in 1) to give the expression vector for the heavy chain. The expression vectors for the light chain and the heavy chain were amplified by PCR, respectively, and recombinantly constructed into the vector pcDNA3.3. Then the transformation, culture and identification were conducted to give the plasmids encoding the amino acid sequence of the antibody P-LCCT_{L}-HC.

### 1.2 Expression of Modified Anti-Human HER2 Antibody P-LCCT_{L}-HC

Transfection was performed using HEK293F seed cells. The plasmids for the light chain and the heavy chain of Pertuzumab were mixed at a mass ratio of 2:1. The plasmid and PEIMAX transfection reagent were diluted with HEK293F basal medium respectively, and then mixed evenly. The mixture was allowed to stand at room temperature, and then added to the seed cell solution for transfection. Samples were analyzed for cell density and viability, and supplemented with HEK293F feed medium (10% volume), and then cooled to 32° C. The incubation was continued. Sampling and analysis for cell density and viability were conducted again at about 72 h and about 144 h.

### 1.3 Purification of Modified Anti-Human HER2 Antibody P-LCCT_{L}-HC

The column was packed with MabSelect Sure ProA, the impurities were washed away with buffer, and the antibody was eluted with the eluent. The eluted antibody was adjusted to pH 5.0 and then subjected to a column packed with Capto S ImpAct. The P-LCCT_{L}-HC antibody was eluted with buffer. The light chain of the antibody P-LCCT_{L}-HC was detected by high resolution mass spectrometry (HR-ESI-MS), theoretical molecular weight: 24208.96, measured: 24204.46.

### 1.4 Preparation of Other Modified Anti-Human HER2 Antibodies

According to a similar method, a terminal modification based on the ligase recognition sequence was introduced at the C-terminal of the light and/or heavy chain of the Pertuzumab, respectively, giving a modified antibody.

The modified anti-human HER2 antibodies described above (SEQ ID NO: 1 to SEQ ID NO: 16) are listed in Table 1. LPETGG in the terminal modification sequence is a recognition sequence of the ligase donor substrate, and GA is a spacer sequence.

TABLE 1

Modified anti-human HER2 antibodies

| | | |
|---|---|---|
| | Sequence | Sequence introduced at the terminal |
| P-LCCT_{L}-HC light chain | SEQ ID NO:1 | GALPETGG |
| P-LCCT_{L}-HC heavy chain | SEQ ID NO:2 | –* |
| P-LC-HCCT light chain | SEQ ID NO:3 | – |
| P-LC-HCCT heavy chain | SEQ ID NO:4 | LPETGG |
| P-LC-HCCT_{L} light chain | SEQ ID NO:5 | – |
| P-LC-HCCT_{L} heavy chain | SEQ ID NO:6 | GALPETGG |
| P-LCCT-HC light chain | SEQ ID NO:7 | LPETGG |
| P-LCCT-HC heavy chain | SEQ ID NO:8 | – |
| P-LCCT-HCCT light chain | SEQ ID NO:9 | LPETGG |
| P-LCCT-HCCT heavy chain | SEQ ID NO:10 | LPETGG |
| P-LCCT-HCCT_{L} light chain | SEQ ID NO:11 | LPETGG |
| P-LCCT-HCCT_{L} heavy chain | SEQ ID NO:12 | GALPETGG |
| P-LCCT_{L}-HCCT light chain | SEQ ID NO:13 | GALPETGG |
| P-LCCT_{L}-HCCT heavy chain | SEQ ID NO:14 | LPETGG |
| P-LCCT_{L}-HCCT_{L} light chain | SEQ ID NO:15 | GALPETGG |
| P-LCCT_{L}-HCCT_{L} heavy chain | SEQ ID NO:16 | GALPETGG |

*: “-” indicates no terminal modification

### 1.5 Preparation of TROP2 Antibody With Ligase Recognition Sequence

A modified anti-human TROP2 antibody was prepared by introducing the amino acid sequence GALPETGG at the C-terminal of the light chain of the antibody described in Table 2-1 by a method similar as 1.4.

TABLE 2-1

Modified anti-human TROP2 antibodies

| | | |
|---|---|---|
| Antibody | Light Chain of Modified Antibody | Heavy Chain of Modified Antibody |
| hRS7 (US 20140120035) | SEQ ID NO: 17 | SEQ ID NO: 18 |
| MAAA1181a (US 20160297890) | SEQ ID NO:19 | SEQ ID NO:20 |

Anti-human TROP2 antibodies Ab0052, Ab0053, Ab0054, Ab0061, Ab0062, Ab0063 and Ab0064 were designed and prepared (Table 2-2). These antibodies each have the amino acid sequence GALPETGG at the C-terminal of the light chain.

TABLE 2-2

Anti-human TROP2 antibodies with ligase recognition sequences

| | | | | | |
|---|---|---|---|---|---|
| Antibody | Sequence of Light Chain | Sequence of Heavy Chain | Antibody | Sequence of Light Chain | Sequence of Heavy Chain |
| Ab0052 | SEQ ID NO: 21 | SEQ ID NO: 22 | Ab0053 | SEQ ID NO: 23 | SEQ ID NO: 24 |
| Ab0054 | SEQ ID NO: 25 | SEQ ID NO: 26 | | | |
| Ab0061 | SEQ ID NO: 27 | SEQ ID NO: 28 | Ab0062 | SEQ ID NO: 29 | SEQ ID NO: 30 |
| Ab0063 | SEQ ID NO: 31 | SEQ ID NO: 32 | Ab0064 | SEQ ID NO: 33 | SEQ ID NO: 34 |

### Example 2 Preparation of Intermediates

### 2.1 Preparation of the Linking Unit

The linking unit LU102 was synthesized by a conventional solid phase polypeptide synthesis using Rink-amide-MBHA-resin or dichloro-resin. Fmoc was used to protect the amino acid and the amino group of the Lk structure in the linking unit. The coupling reagent was selected from HOBT, HOAt/DIC or HATU. After synthesis, the resin was cleaved using trifluoroacetic acid. The product was purified by HPLC, lyophilized and stored for use. Theoretical molecular weight: 538.24, measured: 539.2 [M+H] ⁺.

The linking unit LU104 is prepared according to the above described method, and its structure is as follows:

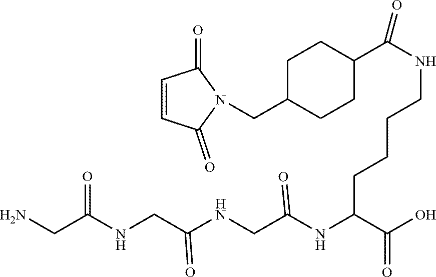 [SMILES: NCC(=O)NCC(=O)NCC(=O)NC(CCCCNC(=O)C1CCC(CN2C(=O)C=CC2=O)CC1)C(=O)O]

The linking units in the following table were prepared according to the above described method. Their structures are as shown hereinabove.

| | | |
|---|---|---|
| Linker | Sequence | Mass spectrometry |
| LU106 | GGG—NH—(C₂H₄—O)₄—C₂H₄—CO—Cys—OH | 540.1 [M+H]⁺ (Theoretical Molecular Weight:, 539.23) |
| LU107 | GGG—Leu—NH—(C₂H₄—O)₄—C₂H₄—CO—Cys—NH₂) | 652.2 [M+H]⁺ (Theoretical Molecular Weight:, 651.33) |
| LU108 | GGG—Gln—NH—(C₂H₄—0)₄—C₂H₄—CO—Cys—NH₂) | 667.3 [M+H]⁺ (Theoretical Molecular Weight:, 666.30) |
| LU109 | GGG—NH—(CH₂)₅—CO—Cys—NH₂) | 405.2 [M+H]⁺ (Theoretical Molecular Weight:, 404.18) |
| LU110 | GGG—(Ac)Lys—Cys—NH₂) | 462.3 [M+H]⁺ (Theoretical Molecular Weight:, 461.21) |

### 2.2 Preparation of Intermediates

The linking unit LU102 and mc-MMAF (molar ratio 1.2:1) were weighed and dissolved in water and DMF, respectively, and then thoroughly mixed to give a mixture, which was reacted at 0-40° C. for 0.5-20 h to obtain intermediate IM102 (ring closed). See formula (vi) above. After purification by HPLC, the molecular weight of the intermediate was analyzed by mass spectrometry. Theoretical: 1462.80, measured: 732.41 [M/2+1]⁺, 1463.81 [M+H]⁺.

As an example, the linking unit LU104 can be transformed into a C-terminal amidated linking unit LU104′ by e.g., (1) protecting the terminal NH₂ of glycine by Boc₂O, (2) reacting the product obtained in (1) with NH₃ in the existence of a coupling reagent, which is selected from HOBT, HOAt/DIC and HATU, and (3) deprotecting the terminal NH₂ of glycine. The C-terminal amidated linking unit LU104’ has the following structure:

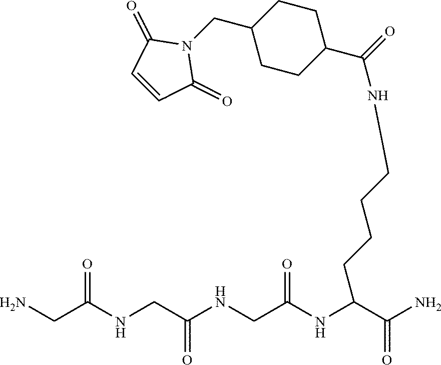 [SMILES: NCC(=O)NCC(=O)NCC(=O)NC(CCCCNC(=O)C1CCC(CN2C(=O)C=CC2=O)CC1)C(N)=O]

Linking unit LU104 and linking unit LU104′ can each independently be used to react with the payload to form a linking unit-payload intermediate.

Then the linking unit LU104′ and DM1 were covalently linked by the above described method to obtain intermediate IM104 (ring closed) having the following structure:

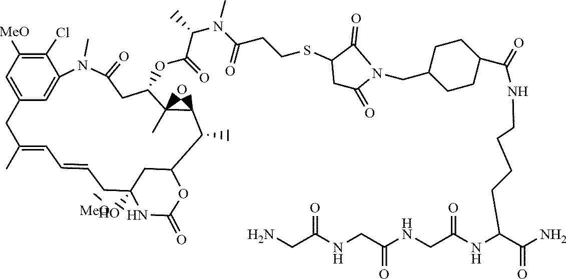 [SMILES: COc1cc2cc(c1Cl)N(C)C(=O)C[C@H](OC(=O)[C@H](C)N(C)C(=O)CCSC1CC(=O)N(CC3CCC(C(=O)NCCCCC(NC(=O)CNC(=O)CNC(=O)CN)C(N)=O)CC3)C1=O)[C@]1(C)O[C@H]1[C@H](C)C1C[C@@](O)(NC(=O)O1)[C@H](OC)/C=C/C=C(\C)C2]

### 2.3 Ring Opening of the Intermediate

IM102 (ring closed) was mixed with an appropriate amount of Tris Base solution or other solution that promotes the ring-opening reaction, and the reaction was performed at 0-40° C. for 0.2-20 h. After the reaction was completed, the product was purified by semi-preparative/preparative HPLC to obtain IM102 (ring open). The structures are shown in the above formula (vii) and formula (vii’). Theoretical molecular weight: 1480.81, measured: 741.41 [M/2+l]⁺, 1481.81 [M+H] ⁺.

The ring opening and purification of the intermediate IM104 (ring closed) was carried out by the above method to obtain the intermediate IM104 (ring open), and the structures are as follows (showed as isomers):

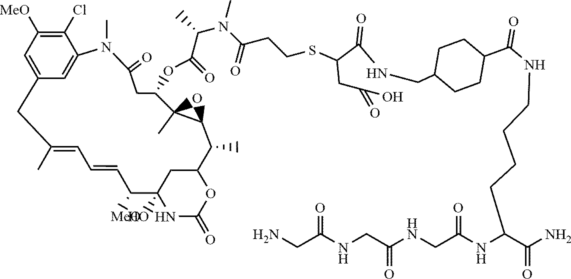 [SMILES: COc1cc2cc(c1Cl)N(C)C(=O)C[C@H](OC(=O)[C@H](C)N(C)C(=O)CCSC(CC(=O)O)C(=O)NCC1CCC(C(=O)NCCCCC(NC(=O)CNC(=O)CNC(=O)CN)C(N)=O)CC1)[C@]1(C)O[C@H]1[C@H](C)C1C[C@@](O)(NC(=O)O1)[C@H](OC)/C=C/C=C(\C)C2]

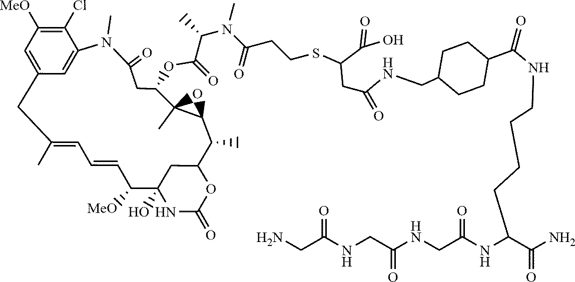 [SMILES: COc1cc2cc(c1Cl)N(C)C(=O)C[C@H](OC(=O)[C@H](C)N(C)C(=O)CCSC(CC(=O)NCC1CCC(C(=O)NCCCCC(NC(=O)CNC(=O)CNC(=O)CN)C(N)=O)CC1)C(=O)O)[C@]1(C)O[C@H]1[C@H](C)C1C[C@@](O)(NC(=O)O1)[C@H](OC)/C=C/C=C(\C)C2]

### Example 3 Preparation of Targeting Molecule-Pharmaceutical Conjugates

### 3.1 Preparation of Conjugate DG102 Targeting HER2 and Reference Drug DG103, Reference Drug DG104

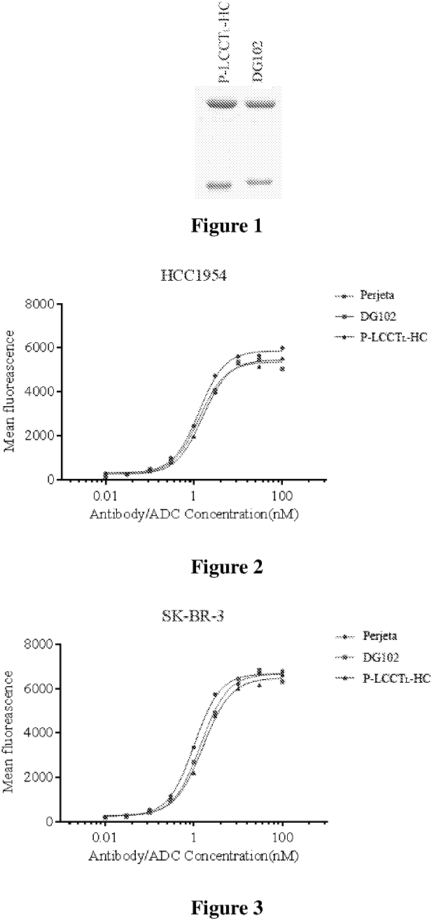

IM102 (ring open) was coupled to the antibody P-LCCT_{L}-HC in a site-specific manner by a ligase to form a drug candidate DG102 having structures as shown in the above formulae (4) and (4′), wherein Formula (4) and formula (4′) are isomers. Specific steps are as follows:
1) Treatment of antibody P-LCCT_{L}-HC
   The antibody P-LCCT_{L}-HC was treated by ultrafiltration, dialysis or desalting column. The storage solution was replaced with a ligase buffer.
2) Enzyme-catalyzed coupling of DG102
   The candidate drug DG102 was prepared by coupling reaction of the antibody P-LCCT_{L}-HC with IM102 (ring open), under the catalysis of a wild type Sortase A or a mutant ligase optimized and engineered based thereon.
   In the ligase buffer, the antibody P-LCCT_{L}-HC and IM102 (ring open) were thoroughly mixed at a molar ratio of 1:1 to 1:100, and added to a solid phase coupling system. The solid phase coupling system comprised a ligase immobilized on the matrix of the solid phase coupling system. The immobilized ligase catalyzed the coupling reaction of the antibody P-LCCT_{L}-HC with IM102 (ring open). The coupling reaction was carried out at 4-40° C. for 0.5-20 h. After the reaction was completed, the reaction mixture was subjected to ultrafiltration or dialysis to remove unreacted intermediate, giving DG102. DG102 was stored at 4° C. or -80° C. in a buffer containing 20 mM citric acid, 200 mM NaCl, pH 5.0.
3) SDS-PAGE detection and analysis of DG102
   The purity and coupling efficiency of DG102 were analyzed by SDS-PAGE. The results are shown in FIG. 1. The coupling reaction occurs at the light chain of the modified antibody P-LCCT_{L}-HC. The light chain of P-LCCT_{L}-HC which is coupled with IM102 (ring open) shows a significant transition in molecular weight compared to that has not undergone a coupling reaction. There is no detectable uncoupled light chain in the coupled product, and the coupling efficiency is as high as 95% or more. The purity of the coupled product meets the expectation.
4) High resolution mass spectrometry (HR-ESI-MS) analysis of DG102
   The light chain of DG102 was analyzed by ESI-MS. The results are as follows: theoretical molecular weight of DG102: 25557.72, measured: 25554.17, confirming the coupling of one cytotoxin molecule at the end of each light chain.
5) HIC-HPLC detection and analysis of DG102
   The DAR distribution of DG102 was analyzed by HIC-HPLC. The antibody P-LCCT_{L}-HC without cytotoxin was less than 5%; the coupled product mainly contains DG102 with DAR of 2, and the DAR of DG102 was calculated to be about 1.84.
6) SEC-HPLC detection and analysis of DG102
   The degree of high molecular weight aggregation of the candidate drug DG102 was analyzed by SEC-HPLC. The results show that no high molecular weight polymer was detected in the candidate drug DG102, indicating that the coupling reaction conditions were mild and did not cause damage to the antibody structure.
7) Preparation of reference drugs
   Using a method similar to 2), the intermediate GGG-Val-Cit-PAB-MMAE was coupled to the modified antibody P-LCCT_{L}-HC in a site-specific manner to give the reference drug DG103, and the structures are as follows (showed as isomers): 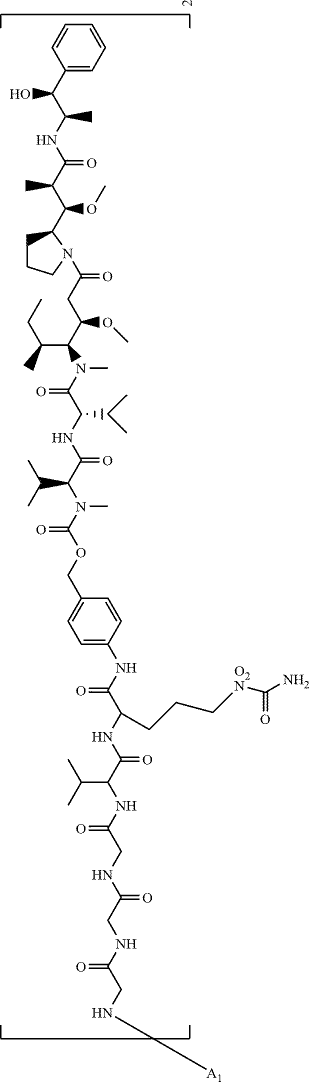
wherein A₁ is P-LCCT_{L}-HC.

Intermediate IM104 (ring open) was coupled to the modified antibody P-LCCT_{L}-HC in a site-specific manner using a method similar to the above point 2) to give the reference drug DG104, and the structure are as follows (showed as isomers):

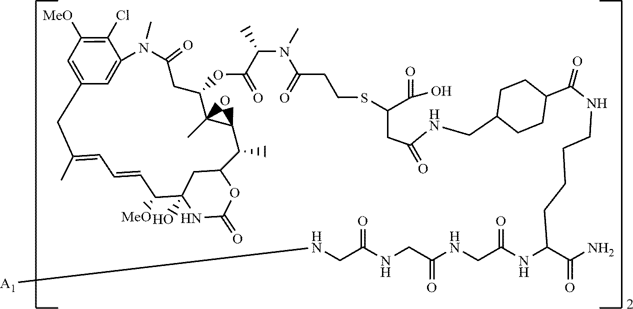

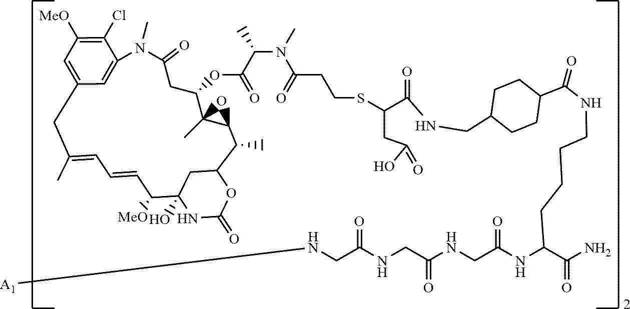

wherein A₁ is P-LCCT_{L}-HC.

### 3.2 Preparation of Conjugates Targeting TROP2

The modified anti-human TROP2 antibodies described above were coupled to the intermediates IM102 (ring open) and IM104 (ring open), respectively, using a method similar to 3.1 to give a TROP2-targeting conjugate. The structure of each fragment in the conjugate are listed in Table 3 below.

TABLE 3

Conjugates Targeting TROP2

| | | | |
|---|---|---|---|
| Conjugate | Antibody | Linker | Payload |
| DG202 | modified hRS7 (SEQ ID NO: 17,18) | LU102 | mc(ring open)-MMAF |
| DG302 | modified MAAA1181a (SEQ ID NO: 19,20) | | |
| DG402 | Ab0052 (SEQ ID NO: 21,22) | | |
| DG502 | Ab0053 (SEQ ID NO: 23,24) | | |
| DG602 | Ab0054 (SEQ ID NO: 25,26) | | |
| DG702 | Ab0061 (SEQ ID NO: 27,28) | | |
| DG802 | Ab0062 (SEQ ID NO: 29,30) | | |
| DG902 | Ab0063 (SEQ ID NO: 31,32) | | |
| DG1002 | Ab0064 (SEQ ID NO: 33,34) | | |
| DG204 | modified hRS7 (SEQ ID NO: 17,18) | LU104’ (ring open) | DM1 |
| DG304 | modified MAAA1181a (SEQ ID NO: 19,20) | | |
| DG504 | Ab0053 (SEQ ID NO: 23,24) | | |
| DG604 | Ab0054 (SEQ ID NO: 25,26) | | |
| DG902 | Ab0063(SEQ ID NO: 31,32) | | |
| DG1004 | Ab0064 (SEQ ID NO: 33,34) | | |

### Effect Example 1 Affinity Determination of Cell Surface ErbB2/Her2 by P-LCCT_{L}-HC and DG102

1) HER2 high-expressing human breast cancer cells HCC1954, SK-BR-3, and HER2 low-expressing cells MCF7 were made into single cell suspensions. 5×10⁵ cells are used for each test. 6.25 nM Pertuzumab, P-LCCT_{L}-HC, and DG102 were added respectively. Incubation was conducted for 60 min at 4° C. Washing was conducted with two repeats: adding 1 ml of PBS washing solution containing 1% BSA, conducting centrifuge at 1000 rpm for 5 min, removing the supernatant.

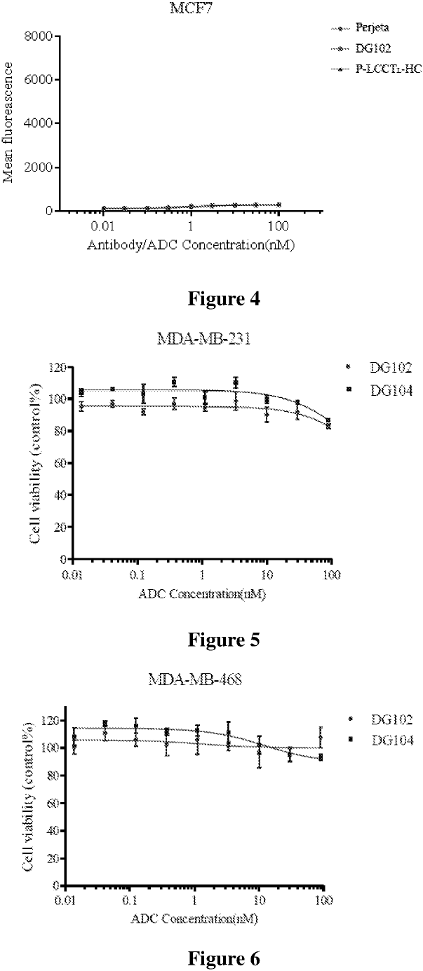

2) To cells incubated with Pertuzumab, P-LCCT_{L}-HC or DG102, 100 µl diluted solution of FITC-goat anti-human IgG antibody was added, respectively. Incubation was conducted for 30 min at 4° C. in the dark. Washing was conducted with two repeats: adding 1 ml of washing solution, conducting centrifuge at 1000 rpm for 5 min, removing the supernatant. The cells were resuspended in 500 µl PBS, passed through a 300 mesh screen, stored on ice in the dark. FACS (fluorescence-activated cell sorting) assay were performed on CytoFLEX S (FIG. 2 to FIG. 4).

The results show that in cells with high or low expression of HER2, affinities of P-LCCT_{L}-HC and DG102 to the ErbB2/Her2 receptor on the cell surface do not show significant difference with that of Pertuzumab, suggesting that according to the present disclosure, no significant influence on the antibody is caused by the modification of the antibody, preparation of the intermediate, or the coupling reaction.

### Effect Example 2 Effect of DG102 on Cell Proliferation

Cytotoxicity experiments were performed using HER2 high-expressing cancer cells HCC1954, SK-BR3, BT-474, and NCI-N87 to analyze the effect of DG102 on tumor cell proliferation.

The IC₅₀ values of DG102 on HER2 high-expressing tumor cells are shown in Table 4:

TABLE 4

| Conjugate | IC₅₀ (nM) | | | |
|---|---|---|---|---|
| | HCC1954 | SK-BR-3 | BT-474 | NCI-N87 |
| DG102 | 0.0108 | 0.007704 | 0.01317 | 0.01173 |
| DG103 | 0.088 | 0.072 | 0.0859 | 0.0876 |
| DG104 | 0.07941 | 0.06098 | 0.1343 | 0.03287 |
| DM1 | 2.673 | 1.919 | 6.241 | 6.970 |
| MMAF | 39.62 | 54.3 | 44.72 | 41.35 |
| mc-MMAF | 116 | 98.64 | 56.1 | 207.6 |
| IM102 (ring closed) | 159.8 | 382.9 | 266.7 | 690.3 |
| IM102 (ring open) | 239 | 597.3 | 492.8 | 940.4 |

The results show that DG102 selectively inhibited the proliferation of a variety of ErbB2/HER2 high-expressing cells. Moreover, the IC₅₀ value of DG102 was significantly lower than those of DG103 and DG104, suggesting that DG102 is more potent. It can be seen that the linking unit of the present disclosure is more advantageous for the cytotoxin to exert pharmacological effects in the cells than Val-Cit-PAB or LU104′. In addition, DG102 which comprises MMAF shows higher activity than DG104 which comprises DM1. In contrast, before conjugation to ADC, free DM1 has higher activity than free MMAF. Therefore, the conjugate of the present disclosure can effectively deliver cytotoxin into the cell, and thus has a crucial and positive effect on the intracellular efficacy of the cytotoxin. Moreover, during the manufacture, the toxicity of the toxin is reduced step by step during the process MMAF mc-MMAF IM102 (ring closed) IM102 (ring open). Accordingly, during the manufacture process, the requirements for protection are reduced and cost is saved. The adverse impact on personnel and environment is also reduced.

Cytotoxicity experiments were performed using human breast cancer cells MDA-MB-231, MDA-MB-468, which are HER2 low-expressing or HER2-negative.

The results of cytotoxicity test of DG102 on HER2 low-expressing tumor cells are shown in FIG. 5 and FIG. 6. The IC ₅₀ values of IM102 are shown in Table 5.

TABLE 5

| Conjugate | IC₅₀ (nM) | |
|---|---|---|
| | MDA-MB-231 | MDA-MB-468 |
| IM102 (ring open) | 2656 | 1812 |
| IM102 (ring closed) | 1712 | 1079 |

The results show that DG102 do not impact the proliferation of HER2-negative or low-expressing tumor cells significantly, indicating low cytotoxicity. Therefore, the conjugate of the present disclosure has high selectivity, and has low toxicity to cells with low expression of HER2, such as normal cells, and the manufacturing process of the drug is less detrimental to the environment and the human body. Furthermore, the IC₅₀ value of IM102 (ring open) is significantly higher than IM102 (ring closed), suggesting that the conjugate of the ring open structure of the present disclosure has lower cytotoxicity in vivo.

### Effect Example 3 Evaluation of in Vivo Efficacy of DG102

Experimental animals: SPF BALB/c nude mice (Shanghai Sippr-BK laboratory animal Co. Ltd., females, 6-8 weeks, weighing approximately 18-22 g, 30 in total.

Tumor cells: Human gastric cancer NCI—N87 tumor cells were cultured and then collected during the logarithmic growth phase. After digestion, they were collected and suspended in an appropriate amount of PBS, pH 7.4.

Experimental method: Each mouse was inoculated with NCI-N87 cells through subcutaneous injection in the right scapular region. The injection volume was 0.2 ml (containing 1 x 10⁷ cells). Each animal in the control group was injected with 0.2 ml PBS, pH 7.4.

The diameters of tumor were measured after 7 days. Animals with tumor ranging from 100 to 200 mm³ were selected and randomized into two experimental groups of 6, namely the PBS control group and the DG102 15 mg/kg group.

14 days after gastric cancer modeling in animals, administration was conducted via tail vein injection. DG102 was formulated at a concentration of 3 mg/ml in PBS, pH 7.4, and 50 µl of the drug was injected per 10 g of body weight.

At each time point of measurement, the average tumor volume and standard error of the mean (SEM) of each group were used for statistical analysis, and one-way ANOVA was used for comparison of tumor volume among groups. The formula for calculating tumor volume is V = 0.5 a × b², wherein a and b are the long and short diameters of the tumor, respectively. All data were analyzed with SPSS 17 software. The standard for statistically significant was P <0.05.

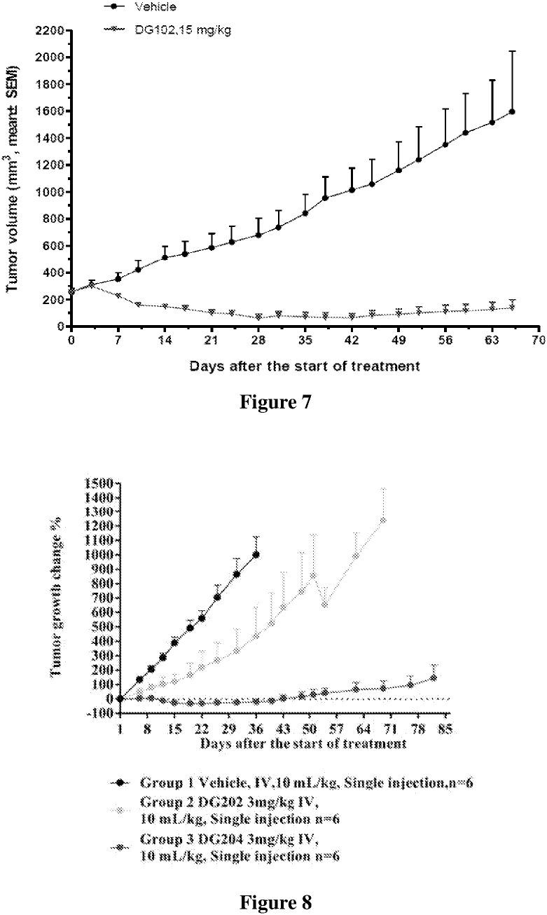

The results showed that DG102 exhibited a good tumor growth inhibitory effect at a dose of 15 mg/kg: a decrease in tumor volume was observed after 10 days of administration, and tumors disappeared in some animals. By 38 days after the start of the treatment, rebound was not observed in tumors growth. The tumor rebounded slightly after 38 days of treatment, but the growth rate was slow. The results showed that DG102 can significantly inhibit the growth of ErbB2/HER2-positive tumors (FIG. 7).

### Effect Example 4 Effect of Conjugate Targeting TROP2 on Tumor Cell Proliferation

A cytotoxicity assay was performed using a method similar as Example 2, TROP2 high-expressing tumor cells NCI-N87, MDA-MB-468, SK-BR-3, and MCF-7 to measure the effects of the TROP2-targeting conjugates DG202, DG302, DG402, DG502, DG602, DG702, DG802, DG902, DG1002 on tumor cell proliferation. The results are shown in Tables 6-1 and 6-2, wherein NCI-N87 and SK-BR-3 are HER2 high-expressing tumor cells, and MDA-MB-468 and MCF-7 are HER2-negative or HER2 low-expressing tumor cells.

TABLE 6-1

| Conjugate | IC₅₀ (nM) | |
|---|---|---|
| | NCI-N87 | MDA-MB-468 |
| DG202 | 0.055 | 0.074 |
| DG302 | 0.070 | 0.125 |
| DG402 | 0.027 | ND |
| DG502 | 0.027 | ND |
| DG602 | 0.028 | 0.058 |
| DG702 | 0.021 | ND |
| DG802 | 0.021 | ND |
| DG902 | 0.021 | 0.036 |
| DG1002 | 0.016 | 0.034 |
| DG204 | 0.108 | 0.262 |
| DG304 | 0.172 | 0.332 |
| DG504 | 0.059 | ND |
| DG604 | 0.065 | ND |

TABLE 6-2

| Conjugate | IC₅₀ (nM) | |
|---|---|---|
| | SK-BR-3 | MCF-7 |
| DG602 | 0.025 | 0.047 |
| DG902 | 0.023 | 0.031 |
| DG1002 | 0.019 | 0.030 |

The results show that when compared with the reference drugs DG204 and DG304, the conjugates DG202 and DG302 of the present disclosure have higher efficacy in TROP2 high-expressing cells. When compared with the reference drugs DG504 and DG604, the conjugates DG202, DG302, DG402, DG502, DG602, DG702, DG802, DG902, DG1002 of the present disclosure have higher efficacy in TROP2 high-expressing cells.

### Effect Example 5 In Vivo Pharmacodynamic Evaluation of TROP2-Targeting Conjugates

The in vivo efficacy study of DG202 was carried out according to the method described in Example 3, and the results are shown in FIG. 8.

The results show that the conjugates of the present disclosure (e.g., DG202, Group3) can significantly inhibit tumor growth in mice at a dose of 3 mg/kg and is more potent than the reference drugs (e.g., DG204, Group2).

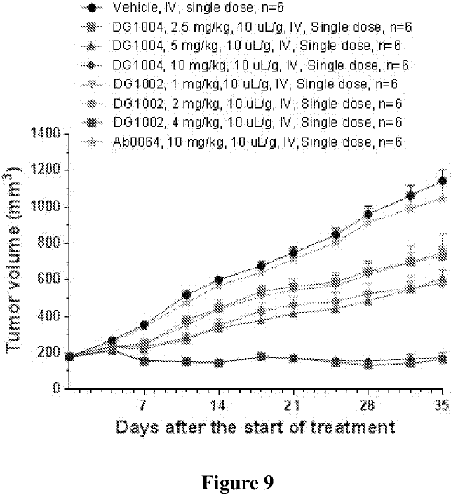

The in vivo pharmacodynamic evaluation of DG1002 was carried out in accordance with the method described in Effect Example 3. The grouping method was as follows: experimental animals with a tumor sizing from 100 to 200 mm³ are selected and then divided into the following groups, each with 6 animals: a control group which received physiological saline; DG1004 2.5 mg/kg group, 5 mg/kg group and 10 mg/kg group which received reference drug DG1004; DG1002 1 mg/kg group, 2 mg/kg group, and 4 mg/kg group which received the conjugate DG1002 of the present disclosure; and Ab2006410 mg/kg group which received antibody Ab0064. After the start of the treatment, tumor sizes were measured twice a week using a caliper, and the results are shown in FIG. 9.

The results show that the conjugates of the present disclosure significantly inhibit tumor growth at lower doses, and such effects are close to those shown by the reference drug at higher doses. For example, results for DG1002 (4 mg/kg) are close to DG1004 (10 mg/kg). Results for DG1002 (2 mg/kg) are close to DG1004 (5 mg/kg). And results for DG1002 (1 mg/kg) are close to DG1004 (2.5 mg/kg). The conjugates of the disclosure have higher efficacies than the reference drugs.

### SEQUENCING LIST

SEQ ID No. 1: P-LCCT_{L}-HC Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 2: P-LCCT_{L}-HC Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG

SEQ ID No. 3: P-LC-HCCT Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

SEQ ID No. 4: P-LC-HCCT Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGLP
ETGG

SEQ ID No. 5: P-LC-HCCT_{L} Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

SEQ ID No. 6: P-LC-HCCT_{L} Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGGA
LPETGG

SEQ ID No. 7: P-LCCT-HC Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECLPETGG

SEQ ID No. 8: P-LCCT-HC Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG

SEQ ID No. 9: P-LCCT-HCCT Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECLPETGG

SEQ ID No. 10: P-LCCT-HCCT Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGLP
ETGG

SEQ ID No. 11: P-LCCT-HCCT_{L} Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECLPETGG

SEQ ID No. 12: P-LCCT-HCCT_{L} Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGGA
LPETGG

SEQ ID No. 13: P-LCCT_{L}-HCCT Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 14: P-LCCT_{L}-HCCT Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGLP
ETGG

SEQ ID No. 15: P-LCCT_{L}-HCCT_{L} Light chain:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 16: P-LCCT_{L}-HCCT_{L} Heavy chain:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGGA
LPETGG

SEQ ID No. 17: hRS7 Modified Light chain

DIQLTQSPSSLSASVGDRVSITCKASQDVSIAVAWYQQKPGKAPKLLIYS
ASYRYTGVPDRFSGSGSGTDFTLTISSLQPEDFAVYYCQQHYITPLTFGA
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 18: hRS7 Heavy chain

QVQLQQSGSELKKPGASVKVSCKASGYTFTNYGMNWVKQAPGQGLKWMGW
INTYTGEPTYTDDFKGRFAFSLDTSVSTAYLQISSLKADDTAVYFCARGG
FGSSYWYFDVWGQGSLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVWDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

SEQ ID No. 19: MAAA1181a Modified Light chain

DIVMTQSPSSLSASVGDRVTITCKASQDVSTAVAWYQQKPGKQPKLLIYS
ASYRYTGVPSRFSGSGSGTDFTLTISSLQPEDFAVYYCQQHYITPLTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQID No. 20: MAAA1181a Heavy chain

QIQLVQSGAEVKKPGESVKVSCKASGYTFTTAGMQWVQQMPGKGLEWMGW
INTHSGVPKYAEDFKGRVTFSLDTSTSTAYLQLSSLKSEDTAVYYCARSG
FGSSYWYFDVWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVWDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

SEQ ID No. 21: Ab0052 Light chain

DIQMTQSPSSLSASVGDRVTITCKASQGINNYLSWYQQKPGKAPKSLIYR
ANRLVDGVPSRFSGSGSGQDYTLTISSLQPEDFATYYCLQYDEFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 22: Ab0052 Heavy chain

QVQLVQSGAEVKKPGSSVKVSCKASGYRFTDYVINWVRQAPGQGLEWMGQ
IYPGSDSFHYNQKFQGRATLTADKSTNTAYMELSSLRSEDTAVYYCARFF
EGLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

SEQ ID No. 23: Ab0053 Light chain

DIQMTQSPSSLSASVGDRVTITCKASQGINNYLSWYQQKPGKAPKSLIYR
ANRLVDGVPSRFSGSGSGTDYTLTISSLQPEDF ATYYCLQYDEFPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGECGALPETGG

SEQ ID No. 24: Ab0053 Heavy chain

QVQLVQSGAEVKKPGSSVKVSCKASGYRFTDYVINWVRQAPGQGLEWMGQ
IYPGSDSFHYNQKFQGRATLTADKSTNTAYMELSSLRSEDTAVYYCARFF
EGLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

SEQ ID No. 25: Ab0054 Light chain

DIQMTQSPSSLSASVGDRVTITCKASQGINNYLSWYQQKPGKAPKSLIYR
ANRLVDGVPSRFSGSGSGTDFTLTISSLQPEDFE1TYYCLQYDEFPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGECGALPETGG

SEQ ID No. 26: Ab0054 Heavy chain

QVQLVQSGAEVKKPGSSVKVSCKASGYRFTDYVINWVRQAPGQGLEWMGQ
IYPGSDSFHYNQKFQGRATLTADKSTNTAYMELSSLRSEDTAVYYCARFF
EGLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

SEQ ID No. 27: Ab0061 Light chain

DIQMTQSPSSLSASVGDRVTITCKASQGINNYLSWYQQKPGKAPKSLIYR
ANRLVDGVPSRFSGSGSGTDFTLTISSLQPEDFATYYCLQYDEFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 28: Ab0061 Heavy chain

QVQLVQSGAEVKKPGSSVKVSCKASGYRFTDYVINWVRQAPGQGLEWMGQ
IYPGSDTFHYNQKFQGRATLTADKSTNTAYMELSSLRSEDTAVYYCARFF
EGLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

SEQ ID No. 29: Ab0062 Light chain

DIQMTQSPSSLSASVGDRVTITCKASQGINNYLSWYQQKPGKAPKSLIYR
ANRLVDGVPSRFSGSGSGTDFTLTISSLQPEDFATYYCLQYDEFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 30: Ab0062 Heavy chain

QVQLVQSGAEVKKPGSSVKVSCKASGYRFTDYVINWVRQAPGQGLEWMGQ
IYPGSDAFHYNQKFQGRATLTADKSTNTAYMELSSLRSEDTAVYYCARFF
EGLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

SEQ ID No. 31: Ab0063 Light chain

DIQMTQSPSSLSASVGDRVTITCKASQGINNYLSWYQQKPGKAPKSLIYR
ANRLVSGVPSRFSGSGSGTDFTLTISSLQPEDFATYYCLQYDEFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 32: Ab0063 Heavy chain

QVQLVQSGAEVKKPGSSVKVSCKASGYRFTDYVINWVRQAPGQGLEWMGQ
IYPGSDSFHYNQKFQGRATLTADKSTNTAYMELSSLRSEDTAVYYCARFF
EGLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

SEQ ID No. 33: Ab0064 Light chain

DIQMTQSPSSLSASVGDRVTITCKASQGINNYLSWYQQKPGKAPKSLIYR
ANRLVSGVPSRFSGSGSGTDFTLTISSLQPEDFATYYCLQYDEFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGALPETGG

SEQ ID No. 34: Ab0064 Heavy chain

QVQLVQSGAEVKKPGSSVKVSCKASGYRFTDYVINWVRQAPGQGLEWMGQ
IYPGSDTFHYNQKFQGRATLTADKSTNTAYMELSSLRSEDTAVYYCARFF
EGLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

1. A compound of formula (I): 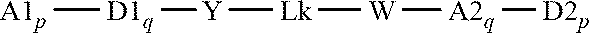
wherein,
D1 and D2 are independently a moiety comprising a recognition sequence of a ligase acceptor or donor substrate;
A1 and A2 are independently a moiety comprising a reactive group which can be coupled with a payload; 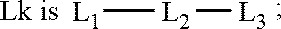
L₁ and L₃ are each independently selected from the group consisting of:
—CH₂—, —NH—, —(CO)—, —NH(CO)—, —(CO)NH—; and a combination of a C₁₋₄ alkylene with one of the following groups: —CH₂—, —NH—, —(CO)—, —NH(CO)—, —(CO)NH—;
L₂ is absent or is a C₇₋₃₄ alkylene, and wherein one or more (—CH₂—) structures in the alkylene is optionally replaced by —O—;
L₁, L₂ and L₃ are each optionally and independently substituted with 1, 2 or 3 substituents selected from —OR₁ and —NR₁R₂;
R₁ and R₂ are each independently selected from the group consisting of hydrogen, -C₁₋₆ alkyl, -(CO)-C₁₋₆ alkyl and -S(=O)₂-C₁₋₆ alkyl;
Y and W are each independently absent or selected from the group consisting of a cleavable sequence, spacer Sp1, and a combination thereof;
the cleavable sequence comprises an amino acid sequence which can be cleaved by an enzyme, and the cleavable sequence comprises 1-10 amino acids;
Sp1 is selected from the group consisting of a spacer sequence containing 1-20 amino acids, PAB, and a combination thereof;
p is 0 or 1, q is 0 or 1, provided that p and q are different.

2. The compound of claim 1, wherein the structure of the compound of formula (I) is as shown in the following formula (1-1) or (I-2): 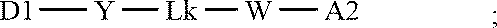 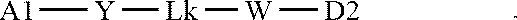
.

3. The compound of claim 1, wherein
L₂ is a C₇₋₃₄ alkylene, wherein one or more of the (—CH₂—) structures in the alkylene can optionally be replaced by —O—, and the alkylene is optionally substituted with 1, 2 or 3 substituents selected from —OR₁ and —NR₁R₂;
preferably, L₂ is selected from groups optionally substituted with 1, 2 or 3 substituents selected from —OR₁ and —NR₁R₂, wherein the said groups are as follows: butylene (—C₄H₈—), pentylene (—C₅H₁₀—), hexylene (—C₆H₁₂—), particularly —C₅H₁₀—.

4. The compound of claim 3, wherein
L₂ is -(C₂H₄-O)ᵢ-C₁₋₄ alkylene;
preferably, L₂ is -(C₂H₄-O)ᵢ-C₁₋₂ alkylene;
more preferably, L₂ is —(C₂H₄—O)ᵢ—C₂H₄—;
wherein i is an integer of 2 to 10.

5. The compound of claim 1, wherein
the cleavable sequence is selected from Phe-Lys, Val-Cit, Val-Lys, GLy-Phe-Leu-Gly, Ala-Leu-Ala-Leu and the combination thereof; and/or
the spacer sequence is Leu or Gln.

6. The compound of claim 1, wherein
the ligase is a Sortase;
preferably, the ligase is Sortase A from Staphylococcus aureus; and/or
the recognition sequence of the ligase donor substrate is LPXTGJ; preferably LPXTG or LPETGG; and/or
the recognition sequence of the ligase acceptor substrate is Gₙ, wherein G is glycine (Gly), and n is an integer of 3 to 10;
X is any natural or unnatural amino acid;
J is absent, or is an amino acid fragment comprising 1-10 amino acids, wherein each amino acid is independently any natural or unnatural amino acid; preferably, J is absent or is Gₘ, wherein m is an integer of 1 to 10.

7. The compound of claim 1, wherein
the reactive group which can be linked to the payload is selected from the group consisting of: amino group, maleimide group, thiol group, pyridyldithio group, haloacetyl group, and isocyanate group; preferably selected from thiol group and amino group; more preferably thiol group.

8. The compound of claim 1, wherein
A1 and A2 are each independently selected from optionally derivatized cysteine;
wherein the derivatization is selected from the group consisting of: 1) amidation of the carboxyl group, the resulting amide being optionally substituted with a C₁₋₆ alkyl group; 2) acylation of the amino group; and 3) linkage of the carboxyl group and/or the amino group to an amino acid fragment comprising 1-10 amino acids or a nucleotide fragment comprising 1-10 nucleotides, wherein the amino acid fragment is preferably Gly.

9. The compound of claim 1, wherein the structure of the compound of formula (I) is as shown in the following formula (I-1-1) or (1-2-1): 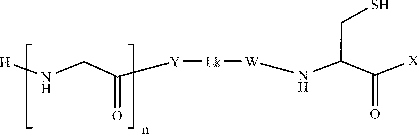 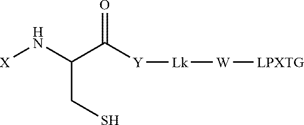
wherein n is an integer of 3 to 10;
x is selected from the group consisting of hydrogen, OH, NH₂, an amino acid fragment comprising 1-10 amino acids, a nucleotide fragment comprising 1-10 nucleotides; preferably,
in formula (I-1-1), x is selected from the group consisting of hydrogen, OH, NH₂, an amino acid fragment comprising 1-10 amino acids, a nucleotide fragment comprising 1-10 nucleotides; especially OH, NH₂ and Gly; and/or
in formula (I-2-1), x is selected from hydrogen, an amino acid fragment comprising 1-10 amino acids, a nucleotide fragment comprising 1-10 nucleotides; especially hydrogen.

10. The compound of claim 1, wherein i is an integer from 2 to 8, for example, 2, 3, 4, 5, 6, 7, 8; preferably 4.

11. The compound of claim 1, selected from 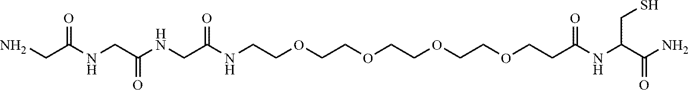 [SMILES: NCC(=O)NCC(=O)NCC(=O)NCCOCCOCCOCCOCCC(=O)NC(CS)C(N)=O] 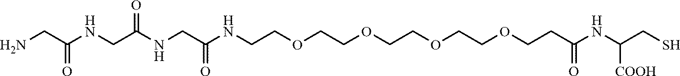 [SMILES: NCC(=O)NCC(=O)NCC(=O)NCCOCCOCCOCCOCCC(=O)NC(CS)C(=O)O] 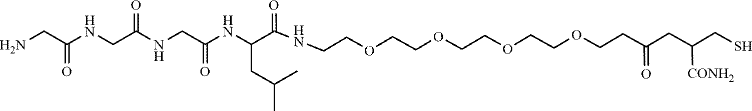 [SMILES: CC(C)CC(NC(=O)CNC(=O)CNC(=O)CN)C(=O)NCCOCCOCCOCCOCCC(=O)CC(CS)C(N)=O] 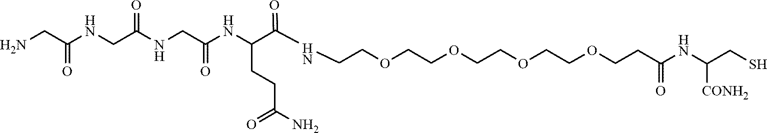 [SMILES: NCC(=O)NCC(=O)NCC(=O)NC(CCC(N)=O)C(=O)NCCOCCOCCOCCOCCC(=O)NC(CS)C(N)=O] 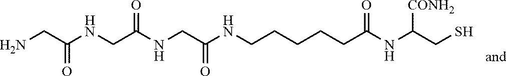 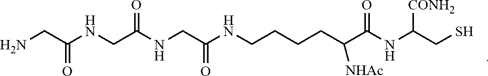
.

12. A compound of formula (II):
(Compound of formula (I))-PLₜ (II) wherein
PL is a Payload, which is linked to the A1 or A2 moiety of the compound of formula (I);
t is an integer of 1 to 20.

13. The compound of claim 12, wherein the structure of the compound of formula (II) is as shown in the following formula (II-1) or (II-2): 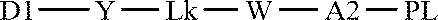 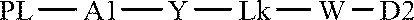
wherein A1, A2, D1, D2, Y, Lk, and W are as defined in any one of the claims 1 to 10, respectively.

14. The compound of claim 12, wherein the structure of the compound of formula (II) is as shown in the following formula (i): 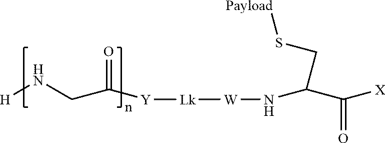
where n is an integer of 3 to 10; 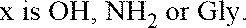
.

15. A conjugate having the structure of formula (III) 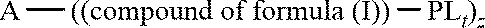
wherein
PL is a Payload, which is linked to the A1 or A2 moiety of the compound of formula (I);
A is a targeting molecule which is linked to the D1 or D2 moiety of the compound of formula (I);
z is an integer of 1 to 20;
t is an integer of 1 to 20.

16. The conjugate of claim 15, wherein
t is 1 and the compound of formula (III) has the structure of the following formula (III-1) or formula (III-2): 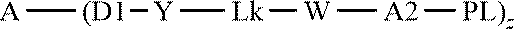 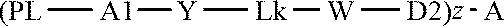
wherein, PL, A1, A2, D1, D2, Y, W, Lk, and z are as defined in any one of the claims 1 to 15, respectively.

17. The conjugate of claim 15, wherein
t is 2-20, the structure of the compound of formula (III) is as shown in any one of the following formula (III-3) to formula (III-6): 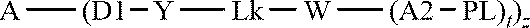 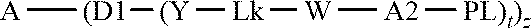 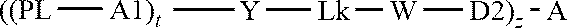 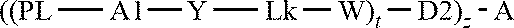
wherein, PL, A1, A2, D1, D2, Y, W, Lk, and z are as defined as defined in any one of the claims 1 to 15, respectively.

18. The conjugate of claim 15, wherein the targeting molecule is an antibody or an antigen binding fragment thereof; the antibody or antigen binding fragment is preferably derivatized to connect with D1 or D2 in the compound of formula (I).

19. The conjugate of claim 18, wherein
the antibody is an anti-human HER2 antibody, preferably selected from PerjetaⓇ, Pertuzumab and Trastuzumab; especially Perjeta®; or
the antibody is an anti-human TROP2 antibody, preferably selected from hRS7, MAAA1181a, Ab0052, Ab0053, Ab0054, Ab0061, Ab0062, Ab0063 and Ab0064.

20. The conjugate of claim 15, wherein
the payload is a cytotoxin or a fragment thereof, with an optional derivatization in order to connect to A1 or A2 in the compound of formula (I);
preferably, the cytotoxin is selected from the group consisting of taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhy-drovinblastine, dolastatin 10 and analogues, halichondrin B, eribulin, indole-3-oxoacetamide, podophyllotoxins, 7-diethylamino-3-(2′-benzoxazolyl)-coumarin (DBC), discodermolide, laulimalide, camptothecins and derivatives thereof, mitoxantrone, mitoguazone, nitrogen mustards, nitrosoureasm, aziridines, benzodopa, carboquone, meturedepa, uredepa, dynemicin, esperamicin, neocarzinostatin, aclacinomycin, actinomycin, antramycin, bleomycins, actinomycin C, carabicin, carminomycin, cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, tiamiprine, thioguanine, ancitabine, gemcitabine, enocitabine, azacitidine, 6-azauridine, carmofur, cytarabine, dideoxyuridine, doxifluridine, floxuridine, calusterone, dromostanolone propionate, epitiostanol, mepitiostane, testolactone, aminoglutethimide, mitotane, trilostane, flutamide, nilutamide, bicalutamide, leuprorelin acetate, protein kinase inhibitors and a proteasome inhibitors; and/or
selected from vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, and pyrrolobenzodiazepine; and/or
selected from auristatins, especially MMAE, MMAF or MMAD.

21. The conjugate of claim 15, wherein the payload is mc-MMAF or mc-Val-Cit-PAB-MMAE, preferably mc-MMAF.

22. The conjugate of claim 15, wherein the conjugate has the structure of the following formula (1): 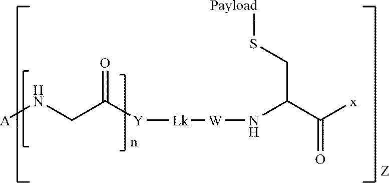
wherein n is an integer of 3 to 10;
x is OH, NH₂ or Gly;
preferably, the conjugate has the structure of the following formula (2) or formula (2′): 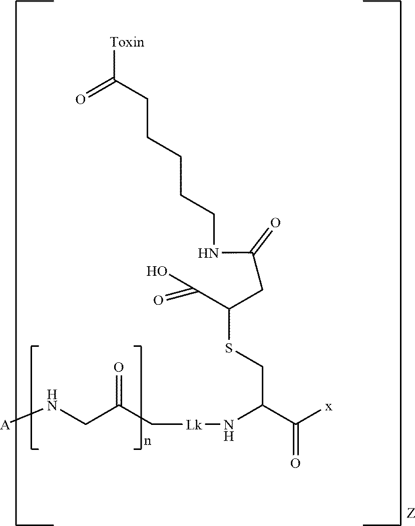 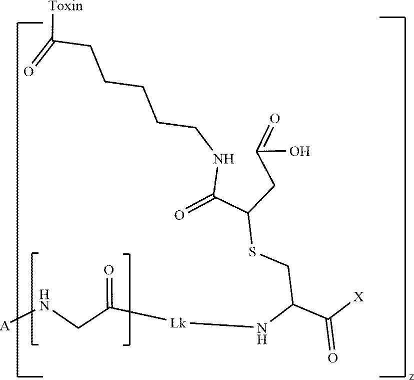
wherein n is an integer of 3 to 10;
x is OH, NH₂ or Gly;
toxin is a cytotoxin;
more preferably, the conjugate has the structure of the following formula (3) or formula (3′): 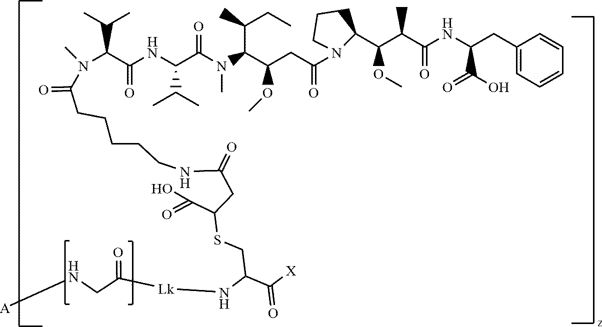 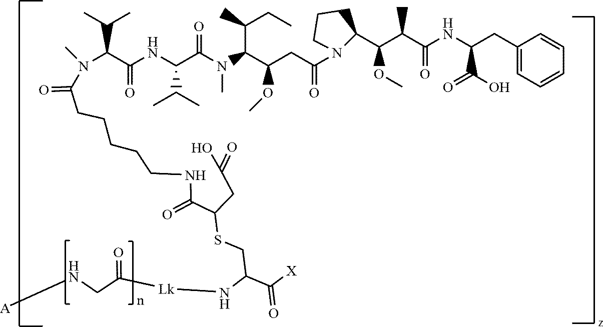
wherein n is an integer of 3 to 10;
x is OH, NH₂ or Gly;
wherein, A and z are as defined in any one of the claims 15 to 21, respectively.

23. The conjugate of claim 15, wherein
the conjugate has the structure of the following formula (4) or formula (4′): 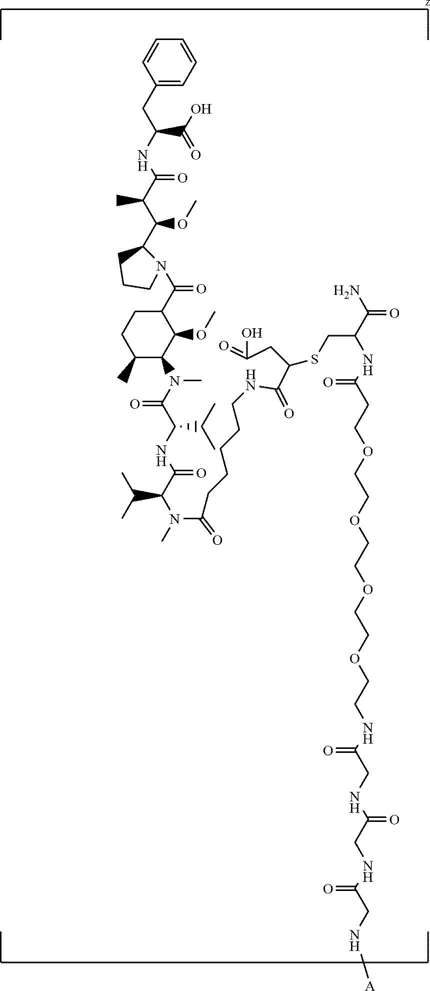 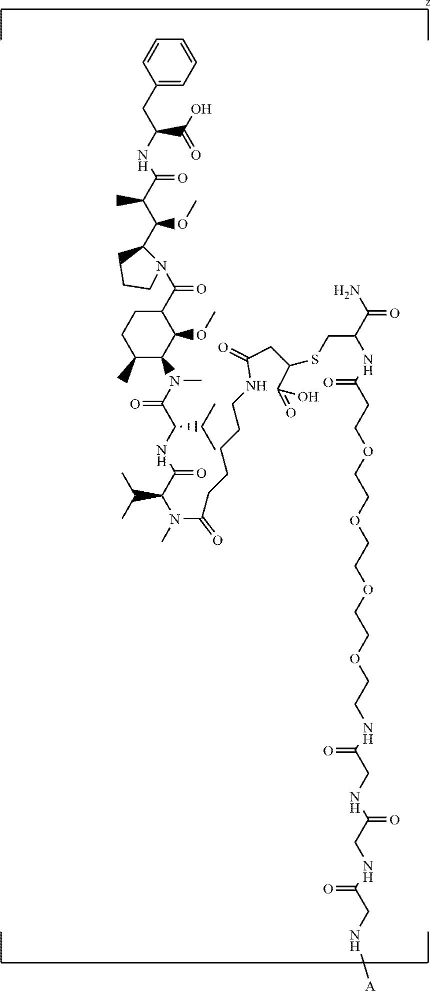
preferably, A is antibody Pertuzumab, hRS7, MAAA1181a, Ab0052, Ab0053, Ab0054, Ab0061, Ab0062, Ab0063 or Ab0064.

24. A pharmaceutical composition comprising a prophylactically or therapeutically effective amount of a conjugate of claim 15, and at least one pharmaceutically acceptable carrier.

25. The pharmaceutical composition of claim 24, wherein the conjugate has a drug to antibody ratio (DAR) of an integer or non-integer of 1 to 20, such as 1-10, 1-8, 1-6, 1-4, 1-3, 1-2.5, 1-2, 1-1.5, 1.5-2 or 1.5-2.5, 1.6-2.1 or 1.8-1.9.

26. A method of treating a disease, comprising administering a pharmaceutical composition comprising a therapeutically effective amount of a conjugate of claim 15 to a subject in need thereof; wherein the disease is a tumor or an autoimmune disease; preferably a HER2-positive tumor or a TROP2-positive tumor;
preferably, the HER2-positive tumor is selected from the group consisting of breast cancer, gastric cancer, lung cancer, ovarian cancer, urothelial cancer; and/or
the TROP2-positive tumor is selected from the group consisting of breast cancer, urothelial carcinoma, lung cancer, liver cancer, endometrial cancer, head and neck cancer, ovarian cancer.
